SEQUENCE LISTING (54 sequences)

SEQ ID NO: 1 - DNA, 517 bp, Zea mays
  CDS (2)...(517)
tgggggggtggggaagacgactctaacacagctcatctacaatgatgaaagagtaaaggagcatttccagttaagggtgtggttgtgcgtttctgaaaattttgacgagatgaagcttaccaaggaaacaattgaatcagttgctagtggattctcatcagccacaacaaacatgaacctgctccaagaagacctctcaaaaaagctgcaaggtaaacggtttcttctagtccttgatgatgtatggaatgaggatcctgaaaaatgggacagatatcgttgtgctctacttagcgggggaaagggaagcaggattataattaccacgcgaaacaaaaatgtggggatactaatgggtgggatgactccttaccatctaaagcagctatcaaacgatgattgctggcagttgttcaaaaaacatgcatttgtagatggtgactccagttcacacccagaattagaaataataggcaaggacatcgtgaagaagttgaaaggcctccccytagcccta

SEQ ID NO: 2 - protein, 172 aa, Zea mays
  VARIANT (1)...(172): Xaa = Any Amino Acid
GGVGKTTLTQLIYNDERVKEHFQLRVWLCVSENFDEMKLTKETIESVASGFSSATTNMNLLQEDLSKKLQGKRFLLVLDDVWNEDPEKWDRYRCALLSGGKGSRIIITTRNKNVGILMGGMTPYHLKQLSNDDCWQLFKKHAFVDGDSSSHPELEIIGKDIVKKLKGLPXAL

SEQ ID NO: 3 - DNA, 634 bp, Zea mays
tgggggggtggggaagacgactcttgccagtgaagtgtaccggaggstcgaagcgcaatttgactacagagctttcgtatcagtgtcacagaaccctgacatgaagaagatattgaggcatatactctgccaccgagggtgtggcggcagccaggaatgggatgagcagcaactcatccacgccgtaagagagttcctccaggataagaggtatgcatgcatcgtatatctgaattcttctcatggcaatcttgccttgaaggaatattatctctctgcagcttcaactcgtcagcagggacatgctttgattaaatttgtgcaaattcgtgctaggtactttgttgtcattgacgatatatggagcacatcagcatggaggattatcagatgtgcttttcctgaaaacaactgttccagtaggatactgacaactactcgcatcgtcacagttgctaagtactgttgctcacctcaccttgaccgtgtgtatgcactggagcctctcgatgcagctcactctgagagcttatttttaagtaggatttttggttccgaagatagatgtcctctccatctgaaagaagcttccgacaaaatactgaaaagatgtggtggcctccccctagcctta

SEQ ID NO: 4 - DNA, 508 bp, Zea mays
  CDS (101)...(508)
tgggggggtggggaagacgactcttgccagtgaagtgtaccggaggstcgaagcgcaatttgactacagagctttcgtatcagtgtcacagaaccctgacatgaagaagatattgaggcatatactctgccaccgagggtgtggcggcagccaggaatgggatgagcagcaactcatccacgccgtaagagagttcctccaggataagaggtactttgttgtcattgacgatatatggagcacatcagcatggaggattatcagatgtgcttttcctgaaaacaactgttccagtaggatactgacaactactcgcatcgtcacagttgctaagtactgttgctcacctcaccttgaccgtgtgtatgcactggagcctctcgatgcagctcactctgagagcttatttttaagtaggatttttggttccgaagatagatgtcctctccatctgaaagaagcttccgacaaaatactgaaaagatgtggtggcctccccctagcctta

SEQ ID NO: 5 - protein, 136 aa, Zea mays
  VARIANT (1)...(156): Xaa = Any Amino Acid
MKKILRHILCHRGCGGSQEWDEQQLIHAVREFLQDKRYFVVIDDIWSTSAWRIIRCAFPENNCSSRILTTTRIVTVAKYCCSPHLDRVYALEPLDAAHSESLFLSRIFGSEDRCPLHLKEASDKILKRCGGLPLAL

SEQ ID NO: 6 - DNA, 498 bp, Zea mays
  CDS (1)...(498)
gggggggtggggaagacgacactaatcagagaactatatgaaaagccaacaacaaagtcaaagcagtttgaatgtcaagcttgggttagcttcccaccgtatttaagttcttcaagcatcctacagttaatccatcaggaattggaggaaacagatacttggtgtcccagaaaacaagtagacaagaaactgcacgaaatattggataaggaccggttcttgctcgtcatagatggagatgttagcaatagtgattgtvatgccatccttgccgcattaccagaaaagaacaacggtagtaggattgtccgtatcatgcaaggtatacgtaagchrccacgtggtattgctgcaaaacactggattgagctgaaatgttttgaaacagaaaaaaccactagcstattcagccaaagggtgtgcatggaagaaaaaataaatattgaaaactttgatgaagttctgcatggtataaccaaaggcctccccctagcctta

SEQ ID NO: 7 - protein, 166 aa, Zea mays
  VARIANT (1)...(166): Xaa = Any Amino Acid
GGVGKTTLIRELYEKPTTKSKQFECQAWVSFPPYLSSSSILQLIHQELEETDTWCPRKQVDKKLHEILDKDRFLLVIDGDVSNSDCXAILAALPEKNNGSRIVRIMQGIRKXPRGIAAKHWIELKCFETEKTTSXFSQRVCMEEKINIENFDEVLHGITKGLPLAL

SEQ ID NO: 8 - DNA, 515 bp, Oryza sp.
  CDS (3)...(515)
tggggggggtggggaagacgactcttgctcaaatggtgtacaatgatgaaagagtcagcaggtactttcaactaaagggctgggtggatgtttctgagggtcattttgacgttaaggcaattgcaagaaaaattatcatgttgttcactaggaatccatgtgatatagaagatatgggtaatctccaaaatatgataacggcgcaagtacaagacatgaagttctttctagttcttgacaatgtgtggaatgtgcagaaggaaatctgggatgctttgctttccctgttggtgggtgctcagttgggaatgatcttattaacaacacgcgatgaaactatttcaaaaatgataggaacaatgccttcttatgatcttagcttcctaacttctgaagaatcatggcagttgttcaagcagatggcctttggatttatagatcaacatatggatcaacagttcgaaggatttggtaggaaaattgtgggaaaatgtggaggcctccccctagcctta

SEQ ID NO: 9 - protein, 171 aa, Oryza sp.
GGVGKTTLAQMVYNDERVSRYFQLKGWVDVSEGHFDVKAIARKIIMLFTRNPCDIEDMGNLQNMITAQVQDMKFFLVLDNVWNVQKEIWDALLSLLVGAQLGMILLTTRDETISKMIGTMPSYDLSFLTSEESWQLFKQMAFGFIDQHMDQQFEGFGRKIVGKCGGLPLAL

SEQ ID NO: 10 - DNA, 506 bp, Oryza sp.
  CDS (3)...(506)
  misc_feature (1)...(506): n = A,T,C or G
tggggggggtggggaagacgacactagctcagaaaatattcaatgataaaaaattagaagggagatttgaccatcgtgcctgggtttgtgtctccaaggagtattctatggtttcccttttgacacaagttcttagtaatatgaaaattcattatgaacaaaatgaatcagttgggaaccttcaaagcaaactcaaagcaggcattgcagacaagagttttttccttgtgttggatgatgtatggcactataaagcatgggaagatttactaagaactccactaaatgcggcagccacaggaataattctagtaactactcgagatgaaactattgctcgtgtaattggggtggaccggactcatagagttgatttgatgtcagccgatgtaggatgggagttactttggaggagcatgaacatcaaagangagaaacaagtgaaaaatctacgggacacaggtatcgagattgtccgcaaatgtggtggcctccccctagcctta

SEQ ID NO: 11 - protein, 168 aa, Oryza sp.
  VARIANT (1)...(168): Xaa = Any Amino Acid
GGVGKTTLAQKIFNDKKLEGRFDHRAWVCVSKEYSMVSLLTQVLSNMKIHYEQNESVGNLQSKLKAGIADKSFFLVLDDVWHYKAWEDLLRTPLNAAATGIILVTTRDETIARVIGVDRTHRVDLMSADVGWELLWRSMNIKXEKQVKNLRDTGIEIVRKCGGLPLAL

SEQ ID NO: 12 - DNA, 518 bp, Oryza sp.
  CDS (3)...(518)
  misc_feature (1)...(518): n = A,T,C or G
tggggggggtggggaagacgactttaacacagctcgtctacaatgatgtgagagtnaagaagcatttccagttaagaatgtggctgtgtgtttctgaaaactttgatgaggcagaacttaccaaggaaacgatagaatcagttgcgagcggattatcatccgccacaacaaacatgaacttgcttcaagaagacctctcaaacaagctgaaaggcaaaaggtttcttctagtattggatgatgtatggaatgaggatcctgatagatgggatagataccgacgtgctctagttgctggtgcaaaaggaagcaaaattatggtgactactcgaaatgaaaatgttgggaaattaatgggcgggttgactccttactatctaaaacagttatcatacaatgatagctggcatttattcataagctatgcatttgtagatggtgactccagtgcacacccaagtttggaaatgatcggcaaggaaattgtccataagttgaaaggcctccccctagcctta

SEQ ID NO: 13 - protein, 172 aa, Oryza sp.
  VARIANT (1)...(172): Xaa = Any Amino Acid
GGVGKTTLTQLVYNDVRXKKHFQLRMWLCVSENFDEAELTKETIESVASGLSSATTNMNLLQEDLSNKLKGKRFLLVLDDVWNEDPDRWDRYRRALVAGAKGSKIMVTTRNENVGKLMGGLTPYYLKQLSYNDSWHLFISYAFVDGDSSAHPSLEMIGKEIVHKLKGLPLAL

SEQ ID NO: 14 - DNA, 510 bp, Oryza sp.
  CDS (1)...(510)
gggggggtggggaagacgacggtagcacagatggtatataatgatgtccgtgttagagaacattttgagcacagtggttggattcatgtctccccaacatttgatgtcctaaggctgacaactgcaataactgagtcattgaccaagagaaactgtggcttcacacagctaagcctagttcatgaggtcctcctcaaagaactggatggaaagaaattattttttgtgcttgatgatgtatggagcgaatgtgaaagttcctggcatgatttaatacgtccactcagctatgctctaacagtcataatattggtaacaacaaggagtaaagaggtggcacgtcttgctggaacagtgaagccattttaccttactgccattcctaatgacgattgctggctattgtttcagcattttgcttttgggaagcaatgtgtgaatgaaaaatcgagtttagttcagattggcaagaaaattttgcagaagtgtggtggcctccccctagcctta

SEQ ID NO: 15 - protein, 170 aa, Oryza sp.
GGVGKTTVAQMVYNDVRVREHFEHSGWIHVSPTFDVLRLTTAITESLTKRNCGFTQLSLVHEVLLKELDGKKLFFVLDDVWSECESSWHDLIRPLSYALTVIILVTTRSKEVARLAGTVKPFYLTAIPNDDCWLLFQHFAFGKQCVNEKSSLVQIGKKILQKCGGLPLAL

SEQ ID NO: 16 - DNA, 506 bp, Sorghum sp.
  CDS (3)...(506)
ttgggggggtggggaagacgactctagcccagaagatctataatgaccataaaataaaaggaagctttagtaaacaagcatggatctgtgtttctcaacaatattctgatatttcagttttgaaagaagtccttcggaacatcggtgttgattataagcatgatgaaactgttggagaacttagcagaaggcttgcaatagctgtcgaaaatgcaagtttctttcttgtgttggatgatatttggcaacatgaggtgtggactaatttactcagagccccattaaacactgcagctacaggaacaattctagtaacaactcgtaatgatacagttgcacgagcaattggggtggaagatattcatcgagtagaattgatgtcagatgaagtaggatggaaattgcttttgaagagtatgaacattagcaaagaaagtgaagtagaaaacctacgagttttaggggttgacattgttcgtttgtgtggtggcctccccctagcccta

SEQ ID NO: 17 - protein, 168 aa, Sorghum sp.
GGVGKTTLAQKIYNDHKIKGSFSKQAWICVSQQYSDISVLKEVLRNIGVDYKHDETVGELSRRLAIAVENASFFLVLDDIWQHEVWTNLLRAPLNTAATGTILVTTRNDTVARAIGVEDIHRVELMSDEVGWKLLLKSMNISKESEVENLRVLGVDIVRLCGGLPLAL

SEQ ID NO: 18 - DNA, 505 bp, Sorghum sp.
  CDS (2)...(505)
tgggggagtggggaagacgacactagctcagaaattatacaatgatcaaagactaaaaggaagctttgagaaacatgcatggatttgtgtttcccagcagtactctcaagttcctttgttgaaagaaatacttcgaaatattggagtgcagcaagagcaaggtgaaagtttgggagagctgaaggccaaacttgcagaagccattaatggaaagagatttttgcttgtgttggatgacttgtgggagtctgatgtatggaccaatttgctaagaactccactcgctgccgctgatcaagtaacaattttagtaacaactcggcatgatacagttgcaaaggcaattggagttggacatatgcaccgagttgaattgctgtcagaggaagtaggatgggagttactatggaagagtatgavcatatctagtgagaaagaagtgctcaacttgcgtgaaactggaattgggattgttcaaaagtgtggtggcctccccctagcctta

SEQ ID NO: 19 - protein, 168 aa, Sorghum sp.
  VARIANT (1)...(168): Xaa = Any Amino Acid
GGVGKTTLAQKLYNDQRLKGSFEKHAWICVSQQYSQVPLLKEILRNIGVQQEQGESLGELKAKLAEAINGKRFLLVLDDLWESDVWTNLLRTPLAAADQVTILVTTRHDTVAKAIGVGHMHRVELLSEEVGWELLWKSMXISSEKEVLNLRETGIGIVQKCGGLPLAL

SEQ ID NO: 20 - DNA, 514 bp, Sorghum sp.
  misc_feature (1)...(514): n = A,T,C or G
tgggggggtggggaagacgacactcacacaacatatatatgaagaagcaaagagccacttccaagtcctggtatgggtatgcgtctctcagaatttcagtgcaagtnagtaggcacaagaaatcgtaaaacaaatccctaaacttgacaatgaaaatggaaatgaaagtgctgaaggtctgattgaaaaaagattgcagtccaaacggttcttgcttgttttggatgacatgtggacagatcacgaagatgaatggaaaaaactgctagccccatttaagaaaatgcaaacaaaaggtaacatggcaatagtcacaacaaggattccgaaggtggcacgaatggtagcaacagtaggctgtcaaataagattagaacggttaagtgatgaagaatgcatgtgtttcttccaagaatgtgtgtttggtaaccgacaaacatgggaagggcatgctattttgcatgatttcgggtacaaaatagtgaagagattgavaggsctccccctagcccta

SEQ ID NO: 21 - DNA, 609 bp, Sorghum sp.
tgggggggtggggaagacgactcttgccaatgaagtgtaccagaagcttgaagggcaatttgactacagagcttttgtgtcagtgtcacaaaaacctgacattaagaagatattgaggcatatactctgccagtatagttgccgagagtgtggcaacaatgaaatatgggatgagcagcaactcatcaacacaacaaggcagctccttaaggataagcggtatgcatcatatatttgtttcttttcatgacaatcttgctttgaaggaatatacatgtgcctatttattgatttgtgcaaattcgtgttaggtactttattgtaattgatgatatatggagcatatcagcatggagaactatcagatgtgcttttcctgaaaataattgttccagtagaatattgacaactactcgcatcatcacagtcgctaagtattgttgctctcctcaccgcgaccatgtttatgaactgaagcctctcgatgcagctcactctaagagcttattttttaatagaatttttggttctgaagatagatgtccccttcatctgaaagaagtttccaatggaatattgaaaaaatgtggtggcctccccttagcccta

SEQ ID NO: 22 - DNA, 517 bp, Sorghum sp.
  CDS (2)...(517)
tgggggggtggggaagacgactcttgccaatgaagtgtaccagaagcttgaagggcaatttgactacagagcttttgtgtcagtgtcacaaaaacctgacattaagaagatattgaggcatatactctgccagtatagttgccgagagtgtggcaacaatgaaatatgggatgagcagcaactcatcaacacaacaaggcagctccttaaggataagcggtactttattgtaattgatgatatatggagcatatcagcatggagaactatcagatgtgcttttcctgaaaataattgttccagtagaatattgacaactactcgcatcatcacagtcgctaagtattgttgctctcctcaccgcgaccatgtttatgaactgaagcctctcgatgcagctcactctaagagcttattttttaatagaatttttggttctgaagatagatgtccccttcatctgaaagaagtttccaatggaatattgaaaaaatgtggtggcctccccttagcccta

SEQ ID NO: 23 - protein, 172 aa, Sorghum sp.
GGVGKTTLANEVYQKLEGQFDYRAFVSVSQKPDIKKILRHILCQYSCRECGNNEIWDEQQLINTTRQLLKDKRYFIVIDDIWSISAWRTIRCAFPENNCSSRILTTTRIITVAKYCCSPHRDHVYELKPLDAAHSKSLFFNRIFGSEDRCPLHLKEVSNGILKKCGGLPLAL

SEQ ID NO: 24 - DNA, 605 bp, Sorghum sp.
  misc_feature (1)...(605): n = A,T,C or G
tgggggggtggggaagacgactcttgcgagggcagtatacgacagccctcaagcaaaggaaaagtttcagtgccgtgcttgggttgctgccaccggtagcgatagctcgccggagcagattaggggtatcctgcgtgatatacaccagcaagttgttccaagagacaccatggattttgacaacaaccatcttgaggcatccctcaaggaatacctcagtgacaagaggtgtgtgcttttgtttatgtttattccatgacaatctgatatctagactatcccaaatgttggtatctatcacttaagctttaaaatcattggttgataggtacttaattgtcattgatgacatccagatggatgaatggagaaccgtaaaatcagtctttgaacacagcagcacaagcagcaggataatattgacgacaactattcagcctatggctaatatgtgcagcagtcatggaaatggttatgtctaccaaatggacacccttggtgaagaagactccaagaaaatagccttkccagggatcaggtcaccngagctggagcatggttcagcagcgctgctcagaaaatgtgatggcctccccctagcccta

SEQ ID NO: 25 - DNA, 505 bp, Sorghum sp.
  CDS (170)...(505)
  misc_feature (1)...(505): n = A,T,C or G
tgggggggtggggaagacgactcttgcgagggcagtatacgacagccctcaagcaaaggaaaagtttcagtgccgtgcttgggttgctgccaccggtagcgatagctcgccggagcagattaggggtatcctgcgtgatatacaccagcaagttgttccaagagacaccatggattttgacaacaaccatcttgaggcatccctcaaggaatacctcagtgacaagaggtacttaattgtcattgatgacatccagatggatgaatggagaaccgtaaaatcagtctttgaacacagcagcacaagcagcaggataatattgacgacaactattcagcctatggctaatatgtgcagcagtcatggaaatggttatgtctaccaaatggacacccttggtgaagaagactccaagaaaatagccttkccagggatcaggtcaccngagctggagcatggttcagcagcgctgctcagaaaatgtgatggcctccccctagcccta

SEQ ID NO: 26 - protein, 112 aa, Sorghum sp.
  VARIANT (1)...(112): Xaa = Any Amino Acid
MDFDNNHLEASLKEYLSDKRYLIVIDDIQMDEWRTVKSVFEHSSTSSRIILTTTIQPMANMCSSHGNGYVYQMDTLGEEDSKKIAXPGIRSXELEHGSAALLRKCDGLPLAL

SEQ ID NO: 27 - DNA, 1040 bp, Sorghum sp.
  misc_feature (1)...(1040): n = A,T,C or G
tgggggggtggggaagacgacattggcaaaagaggtcagtcacaagntccaaggcatttcgattgtcatgcttttgtctctgtctcgcaacagccgaatgttaagaaaattatgaaggatgtgatctctcaagtgccttgcaaaaaggattttacagaagatatcgacacctgggatgaaaagaaatttattgggaagcttagagaactgttacaagataagaggtaatttcatgccccctttttttagaaaagaagaatttattagttttgctccttttagtgttcaaaaaacctaaatataccacagatcttgtaaatatgagaataatagaatagttgtctaagtggaatatatttttttgtaatagacaaacttatctgttgacttaatatttcctaatacagtagttatgctaaggtcttgtttacttccatccaaaacccaaaaattttcaagattctccgtcatatcgaatctttagacgcatgccwtggggtattaaatwtagmcgaaataaaagctaattgcacagtttggtcgaaatttacgagacgaatcctttgagcctagttagtccatgttgaatttattttatcatgttcttcaaattctatattttttcatgtatggcacttgaattacttagcatggtgtaggagtctcatggtatgtctgtatttttttttctacttaaactccttcccataattggttaacaatatttatttatttctatcaggtacctcatcattattgatgatatatggtctatactggcatgggacgctattaaatatgctttcccagagaataatttttctagcagaattatagcaactacacgcatcgttgatgtagcgaggtcatgttgtctcggtggtaatgatcgcatgtacgagatggaagctctaagtggtcttcactcgaaaaaattatttttcaagagaacatttggatctgaagactgttgcccggatgtgctaaaagaagtatcaaatgaaatattgaaaaaatgtggaggcctccccttagcccta

SEQ ID NO: 28 - DNA, 522 bp, Sorghum sp.
  CDS (112)...(522)
  misc_feature (1)...(522): n = A,T,C or G
tgggggggtggggaagacgacattggcaaaagaggtcagtcacaagntccaaggcatttcgattgtcatgcttttgtctctgtctcgcaacagccgaatgttaagaaaattatgaaggatgtgatctctcaagtgccttgcaaaaaggattttacagaagatatcgacacctgggatgaaaagaaatttattgggaagcttagagaactgttacaagataagaggtacctcatcattattgatgatatatggtctatactggcatgggacgctattaaatatgctttcccagagaataatttttctagcagaattatagcaactacacgcatcgttgatgtagcgaggtcatgttgtctcggtggtaatgatcgcatgtacgagatggaagctctaagtggtcttcactcgaaaaaattatttttcaagagaacatttggatctgaagactgttgcccggatgtgctaaaagaagtatcaaatgaaatattgaaaaaatgtggaggcctccccttagcccta

SEQ ID NO: 29 - protein, 137 aa, Sorghum sp.
MKDVISQVPCKKDFTEDIDTWDEKKFIGKLRELLQDKRYLIIIDDIWSILAWDAIKYAFPENNFSSRIIATTRIVDVARSCCLGGNDRMYEMEALSGLHSKKLFFKRTFGSEDCCPDVLKEVSNEILKKCGGLPLAL

SEQ ID NO: 30 - DNA, 1044 bp, Sorghum sp.
  misc_feature (1)...(1044): n = A,T,C or G
tgggggggtggggaagacgacattggcaaaagaggtcagtcacaagatccaaggcatttcgattgtcatgcttttgtctctgtctcgcgacagccgaatgttaagaaaattatgaaggatgtgatctctcaagtgccttgcaaaaaggattttacagaggatatcgacacctgggatgaaaagaaatttattgggaagcttagagaactgttacaagataagaggtaatttcatgccccctttttttagaaaagaagaatttatttgttttgctctttttagtgttcaaaaaaattaaatataccacagatcttggtaaatatgrgawtaatagaattagttgtctaagtggaatatattttttkgtaatagacaaacttatctgttgacttaatatttcctaatacagtagttatgctaaggtcttgtttacttccatccaaaacccaaaaattttcaagattctccgtcatatcgaatctttagacgcacgcatggagtattaaatatagacgaaaataaaagctgattgcacagtttggtcgaaatttacgagacgaatcttttgagcctagttagtccatgttgaatttattttatcatgttcttcaaattctatattttttcatgtatggcacttgaattacttagcatggtgtaggagtctcatggtatgtctgtattttttttttctacttaaactccttcccataattggttaacaatatttatttatttctatcaggtacctbntcattattgatgatatatggtctatactggcatgggacgctattaaatatgctttcccagagaataatttttctagcagaattatagcaactacacgcatcgttgatgtagcaaggtcatgttgtctcggtggtaatgatcgcatgtatgagatggaagctctaagtgatcttctctcgaaaaaattatttttcaagagaacatttggatctgaagactgttgcccggatgtgctaaaagaagtatcaaatgaaatattgaaaaaaatgtggaggcctccccttagcctta

SEQ ID NO: 31 - DNA, 1038 bp, Sorghum sp.
  misc_feature (1)...(1038): n = A,T,C or G
tgggggggtggggaagacgacattggcaaaagaggtcagtcactagatccaaggcatttcgattgtcatgcttttgtctctgtctcgcaacagccgaatgttaagaaaattatgaaggatgtgatctctcaagtgccttgcaaaaaggattttacagaagatatcgacacctgggatgaaaagaaatttattgggaagcttagagaactgttacaagataagaggtaatttcatgccccctttttttagaaaagaagaatttattagttttgctctttttagtgttcaaaaaaattgaatataccacagatcttgtaaatatgagaataatagaatagttgtctaagtggaatatatttttttgtaatagacaaacttatctgttgacttaatatttcctaatacagtagttatgctaaggtcttgtttacttccatccaaaacccaaaaattttcaagattctccgtcatatcgaatctttagacgcatgcmtggrgtwttaaatatagacgaaaataaaagctaattgcaccrgtttggtcgaaatttacgagacgaatcttttgagcctwgttagtccatgttgaatttattttvtcatgttcttcaaattctatattttttcatgtatggcacttgaattacttagcatggtgtaggagtctcatggtatgtctgtatttttttttctacttaaactccttcccataattggttaacaatatttatttatttctatcaggtwcctcntcactattgatgatatatggtctatactggcatgggacgctattaaatatgctttcccagagaataatttttctagcagaattatagcaactacacgcatcgttgatgtagcaaggtcatgttgtctcggtggtaatgatcgcatgtatgagatggaagctctaagtgatcttcactcgaaaaaattatttttcaagagaacatttggatctgtagactgttgcccggatgtgctaaagagtvtcaatgaatattgrgaaatgtgggcctcccctgccttagcctta

SEQ ID NO: 32 - DNA, 1043 bp, Sorghum sp.
  misc_feature (1)...(1043): n = A,T,C or G
tgggggggtggggaagacgacattggcaaaggaggtcagtcacaagatccaaggcatttcgattgtcatgcttttgtctctgtctcgcaacagccgaatgttaagaaaattatgaaggacgtgatctctcaagtgccttgcaaaaaggattttacagaagatatcgacaactgggatgaaaagaaatttattgggaagcttagagaactgttacaagataagaggtaatttcatgccccctttttttagaaaagaagaatttattagttttgctctttttagtgttcaaaaaaattaaatataccacagatcttgtaaatatgrgawtawtrgaatagttgtctaagtggaataawatttttttgtaatagacaaacttatctgttgacttaatattycctaatacagtagttatgctaaggtcttgtttacttccatccaaaacccaaaaatttttcgagattctccgtcatatcgaatctttagacgcatgcatggagtattaaatatagacgaaaataaaagctaattgcacagtttggtcgaaatttacgagacgaatcttttgagcctagttagtccatgttgaatttattttatcatgttcttcgaattctatattttttcatgtatggcacttgaattacttagcatggtgtaggagtctcatggtatgtctgtattttttttttctacttaaactccatcccataataggttaacaatatttatttatttctatcaggtacctcmncattgttgatgatatatggtctatactggcatgggacgctattaaatatgctttcccagagaataatttttctagcagaattattgcaactacacgcatcgttgatgtagcaaggtcatgttgtctcggtggtaatgatcgcatgtatgagatggaagctctaagtgatcttcactcgaaaaaattatttttcaagagaacatttggatctgaagactgttgcccggatgtgctaaaagaagtatcaaatgaaatattgaaaaaatgtggaggcctccccctagcccta

SEQ ID NO: 33 - DNA, 6760 bp, Artificial Sequence
  Sorghum sp.  gene in BAC clone
aaaaaattactaactatacctgtaatttgcgacataaatcttttaaatctagttagtccatagttagrttaccattmccnaataaaacaaaatttcaagattttttcacctcttttgggtgtcatatcgaatgtttcgtagaacatcggaagaggtctttggatactaataaaaaaactaattacataactttgctgtaaactacaagacgaatttattaagtctaattaatctattattagtacatgtaagttactatatgatttccaactatactatatacttaggccttgtttagttcccaccaaaatacaaaaagttttcaagattcactgtcacatcgaatctttgcggcacatgcatggagcatgaaatatagataaaaaaataaataattgcacaatttacctgtaaattgcgaaatgaatcttttgaatctaggtagtacataattgaataatatttgccaaatacaaacgaaagtgttacaggagcgaaattcaaaaaaatttctcaactaaccaagatcttagtttattttttatctatatttaatactcaatgcatgcgctaaacattcaatgtgatgattgaaaacttaggacatgagacgtatttgggaacccaaatctaaaatgaatctctaacacaataactatagcctccatagagtaaccatatagaagactcattttgggtatcaggagaggcataaatccaaatttgggtaccctctctcctcgagacccatttgtagagaatgttgtcttttaggtcttgttgaagaagactaaaaataggtatagagcgctacacaaaagacaaatgagtcttatattttgggtgatgattgttgggagaaaatggctattataggactccaatcgttgtggcttctctcaaatagaactctaaatatttgtttctcccaaataggactctaaacattgtttgtaagctctaataacattttaatactattaacccatgttttaagtctttaaatatatgtatttgaccatgacatgactcttttgcccctgggaccattgtgccgccgacatagaaccacggcgagacgactctcactttgccaccggagaacgcgttcatggccaccggagggagaaggaacccatcgagcgccagccccgcgaacgaggcgaggtcctcccatagcgtagctggctctggggccaacacgaacggcgagagaagtcctcccatgcagcgcagcakcagacatatcgctcctctcggctgccgccgccgctgtcccgtccttgctctgtcggcagccgagctccgagcagaccaagctagttgaaggaggcatagctgaagcacgaacgccatcagcgtgggcaccctcagcatcgtctgcttcagctcgtacgacacttttctcttggcgttggaggatgtgtagggccgtgaagatgcacggcagcgtcgtggagacaaccagcagcatgacactctccaggtccatccgcgagatggatgcggacaccggcctgctcaacgtacatcccgagtagagcagtaaagtccaggggcaaaagagtcaagccatggccaaatacatatatttmaagacttaaaacatggctaataatattaaaatgttattagagcttacaaagaatgtttagagttctatttgagagaaacaaatatttggagtcctatttagaggtcatataataaccattttgtcttagatttgggaacgaaaaaaggccttaccccgcaaggcctccaattgcctccctctctctctttcvctctctctccggccacggtcgtcgctgcgccgccgcacagaggccggggcgtggacgctacacgcatccgtytctcctcctgstggccgcgctgtcttcctattctccaagggcttgattgggggaatgtgatgtgataccgtcagcgatggtccgtcaaccgacagggagatttcgtcgggcgtctggcgggctggaccaacgagacgacgaccggccacttctttctcgctgggcggctcaagaaatcgcaggtttccacgcagtccctgtcgttcgggctcaaattgtcgttacgtcacaggcgagtagttctttgactaatctttgattgatttggcagttgactgttgcagtttgatatctgatagtgatttgtccgcaggcggtggctcaactgctcggtgagagtgaaatatggctaatcgaggccctttacttgctttcctgttgacgacttgagtagccttgaagtttgcagtagtgtgccttgtgcaattccgcacttcttttcctatgatttcataagtgcttcatgtctttaatttaatcttgacagattcagtgatctttcaaatctgctctgtgcaatttgagaaccccctgtcttttggcatgaccgcacactagctagacaaggactgctgcttagaggtcacattattactagctagttgttttcctcccttttattggagagtgctcatcttttccttgctcatgtgtgcttggcagtaagcatctttaagaggctatactccctaaacctgctcctttggaaggcactatatttgctaatttatttagcattgtttagcttgttggaaactggggctgattggtatgaagtataaacaatgtgaaataatattctagtctgtactgctcaaacaactctcactgtcctatgttgaattataatttttttcaggtcatgggcgttcatattgtcacatattactgaaaaaatgggccactgtggaaacaggtccagtttacatagtagtcatcttagtagacactgacagatcctctagtggtttgactaatcatagattcccttaagaaaatctttacaactttgctctaaacctatcaccttatttatctattctttttaatcttaaggttccatgtcctttccctgaatattataatttaggtatatgacctgtgatgtttgtcttctgcattcacctaaattatttttagggatatacaaatcttttatcattaggtggcttcactagtaagacacacattttatcagctttatcttctaactagttgattgaccttgcaatgtgggtgattcacacttttttcacacttgcattcttgaccacttgactagtcttgaaatgctttttgtgcacctttttttatgcatcaaggaagtgattgttgtgtacaccttacctggcttacctgcttactccttttattctgttcttatacttcagtcgcttccttttcttgtgcatgtggagttgacttactgagcatgtataagaagagccctcagacatgaaagttgcacaggcacaaatactgacaccagaataccagatgcagtgaggggtgggggatggaagctattgtgtgtgcatcacatggagccatgggctccttgctgtggaagctgggtgcattgctctctgatgagtacaagcttctaagtagtgtgaaggtggatatgatgttccttaaagctgagcttgaggtcatgcatgctttcctgaagaagatgtcggaggtggaggttcctgatgagcagtccaagtgctggatgaaggaggtgcgggagctgtcctacgacattgaggacagcatcgacagtttcatattttctcttggttgtgagtccaacagcgaacctagaggcttcaaggggtttgttggtaggtgcttgaacttgtttgccgatgctaagacacgtcattggattgccaagaagatccaacgtctcaaatgccatgttatagaggctagcaatcggcgtgggaggtacagggttgatgatgctgtccccagattgagtagaacaagcatagaccctcgcttgccggcattgtacactgagacaacaaggcttgttggtgttgatggcccaagggacaaacttatcaagttgctaacagaaagagagggcacaacgacacagctgagtgtggtctccattgttggatttggtgggcttgggaagactactcttgccaatgaagtgtaccagaagcttgaagggcaatttgactacagagcttttgtgtcagtgtcacaaaaacctgacattaagaagatattgaggcatatactctgccagtatagttgccgagagtgtggcaacaatgaaatatgggatgagcagcaactcatcaacacaacaaggcagttccttaaggataagcggtatgcatcatatatttgtttcttttcatgacaatcttgctttgaaggaatatacatgtgcctatttattgatttgtgcaaattcgtgttaggtactttattgtaattgatgatatatggagcatatcagcatggagaactatcagatgtgcttttcctgaaaataattgttccagtagaatattgacaactactcgcatcatcacagtcgctaagtattgttgctctcctcaccgcgaccatgtttatgaactgaagcctctcgatgcagctcactctaagagcttattttttaatagaatttttggttctgaagatagatgtcctcttcatctgaaagaagtttccaatggaatattgaaaaaatgtggtggcttaccattggcaatcattaccgtagctagtttattggtcactaaagctattacaaaagaagaatgggagaagatgctgaagtctattggttcagcacttgaaaaagatacagatatggaagagatgaagaagattcttttgcttagttacaatgatctcccctaccacttgaagacatgcttgttatatctaggtgtgtttcctgaagattatgagattaagagggatcgactgataagaagatggattgctgaaggttttatcactacagaaggtggacaagacatggaggaaataggagaatgctatttcaatgaacttatcaacaggagcatgattcagcctgttggaattcagtatgatggtcgggctgatgcttgccgtgtccatgatatgattcttgatctcatcatatccaaatcagttgaagaaaactttctaaccttatgtggtgatggaaatcacaagctgttgcaacaggataaggttcgtcggctatccatcaattatcatgctcgagatgatattatagtaccaacaaatatgattgtttctaatgttcgatccctcactatatttgggtatgatgagaatatgcctggtctttccaactttctactcttgcgagttctggatctagaaaatagagtggtgctggaatacaattaccttagacacataggtaggctttctcagttgaggtacctccgactcagttcaagaagaataactgcacttcctgaacaaataggagatctacaaaacttgcagaccttagatctgaggtggacaaggattaaaagattgccacagagtgttgtcctcctacgacgattgacatgcttgttggttaacagtttagaattgcctgaaggaattggaaatatgcaagctctacaggagttatcagagattgaaattaactgtcatacatcagtgtcctctttgctggaactgggcaaattgactaatataagaattcttgggctaaattggtgcatcctcgatacaaattatgtaacaaaaatccatgcagatagtttggttatgtccctctgcaaattaggcatgctcaatcttcgatcgatacagattcaaagctatcatagttgttcccttgatttcttaaaggattcttggtttcctcctcctcgtcgcctccaaatatttgatatgtccatagattaccatttccccagaattccaaactggataatctcactggagtacctcagttacctagacatatatcttactccagtggatgaggaatcatttcgaaccctgggggatttgccctctctgttgttcctctggatatcctcaagagaagcaaagcctaaagaaggggttattgtcagcagcaatggtttccgctgtctgaaggagttctacttcacctgttgggaaattggtacagggctgtcttttgaaccaggagccatgccaatgcttgaaaaactgcggattccattcaacgcgcatggtgtgtgctctttgcacggtgttctggattctggaatctggcacctctgttccctaaggcatctccatgtcgagattatttgtcatggtgcgaggcttaaggaggtggaggccgtggaggaagctgtcaagaatgcagccagctacctttctgatgagctcttccttgatgtaagaagatgggacgaagaagagattttgaaggacgaggagcataaactggaggaagaggagtttagttcttatgattcaaaaagcataactcaatactgaagtttgatttcttctcttaatcacaagcatgcacatgtatttctgtttctactgactagcacattgtagggtgcctacactattgactttagcgcttgtgaagagctgattcggcacgatcatgattcaaacaaattagtggtccattatattcagtgacacaataatttgttcttcctttttcaaatttgaagtttgggaaaatatactagatgcaactgcaagcattttggaacccctgctcctctgattacttggtccttaaattctcctctaattgcttgaaggcttgcttgtccttcaaatctgaagctttctgtccttcaaatctgaagctttctaaaagctggttaccgtggtccccggggactccttcaaatttgaagtttttttttctcgaatccttcaaattcgaatcaagaactgtctgc

SEQ ID NO: 34 - DNA, 2954 bp, Sorghum sp.
atggaagctattgtgtgtgcatcacatggagccatgggctccttgctgtggaagctgggtgcattgctctctgatgagtacaagcttctaagtagtgtgaaggtggatatgatgttccttaaagctgagcttgaggtcatgcatgctttcctgaagaagatgtcggaggtggaggttcctgatgagcagtccaagtgctggatgaaggaggtgcgggagctgtcctacgacattgaggacagcatcgacagtttcatattttctcttggttgtgagtccaacagcgaacctagaggcttcaaggggtttgttggtaggtgcttgaacttgtttgccgatgctaagacacgtcattggattgccaagaagatccaacgtctcaaatgccatgttatagaggctagcaatcggcgtgggaggtacagggttgatgatgctgtccccagattgagtagaacaagcatagaccctcgcttgccggcattgtacactgagacaacaaggcttgttggtgttgatggcccaagggacaaacttatcaagttgctaacagaaagagagggcacaacgacacagctgagtgtggtctccattgttggatttggtgggcttgggaagactactcttgccaatgaagtgtaccagaagcttgaagggcaatttgactacagagcttttgtgtcagtgtcacaaaaacctgacattaagaagatattgaggcatatactctgccagtatagttgccgagagtgtggcaacaatgaaatatgggatgagcagcaactcatcaacacaacaaggcagttccttaaggataagcggtatgcatcatatatttgtttcttttcatgacaatcttgctttgaaggaatatacatgtgcctatttattgatttgtgcaaattcgtgttaggtactttattgtaattgatgatatatggagcatatcagcatggagaactatcagatgtgcttttcctgaaaataattgttccagtagaatattgacaactactcgcatcatcacagtcgctaagtattgttgctctcctcaccgcgaccatgtttatgaactgaagcctctcgatgcagctcactctaagagcttattttttaatagaatttttggttctgaagatagatgtcctcttcatctgaaagaagtttccaatggaatattgaaaaaatgtggtggcttaccattggcaatcattaccgtagctagtttattggtcactaaagctattacaaaagaagaatgggagaagatgctgaagtctattggttcagcacttgaaaaagatacagatatggaagagatgaagaagattcttttgcttagttacaatgatctcccctaccacttgaagacatgcttgttatatctaggtgtgtttcctgaagattatgagattaagagggatcgactgataagaagatggattgctgaaggttttatcactacagaaggtggacaagacatggaggaaataggagaatgctatttcaatgaacttatcaacaggagcatgattcagcctgttggaattcagtatgatggtcgggctgatgcttgccgtgtccatgatatgattcttgatctcatcatatccaaatcagttgaagaaaactttctaaccttatgtggtgatggaaatcacaagctgttgcaacaggataaggttcgtcggctatccatcaattatcatgctcgagatgatattatagtaccaacaaatatgattgtttctaatgttcgatccctcactatatttgggtatgatgagaatatgcctggtctttccaactttctactcttgcgagttctggatctagaaaatagagtggtgctggaatacaattaccttagacacataggtaggctttctcagttgaggtacctccgactcagttcaagaagaataactgcacttcctgaacaaataggagatctacaaaacttgcagaccttagatctgaggtggacaaggattaaaagattgccacagagtgttgtcctcctacgacgattgacatgcttgttggttaacagtttagaattgcctgaaggaattggaaatatgcaagctctacaggagttatcagagattgaaattaactgtcatacatcagtgtcctctttgctggaactgggcaaattgactaatataagaattcttgggctaaattggtgcatcctcgatacaaattatgtaacaaaaatccatgcagatagtttggttatgtccctctgcaaattaggcatgctcaatcttcgatcgatacagattcaaagctatcatagttgttcccttgatttcttaaaggattcttggtttcctcctcctcgtcgcctccaaatatttgatatgtccatagattaccatttccccagaattccaaactggataatctcactggagtacctcagttacctagacatatatcttactccagtggatgaggaatcatttcgaaccctgggggatttgccctctctgttgttcctctggatatcctcaagagaagcaaagcctaaagaaggggttattgtcagcagcaatggtttccgctgtctgaaggagttctacttcacctgttgggaaattggtacagggctgtcttttgaaccaggagccatgccaatgcttgaaaaactgcggattccattcaacgcgcatggtgtgtgctctttgcacggtgttctggattctggaatctggcacctctgttccctaaggcatctccatgtcgagattatttgtcatggtgcgaggcttaaggaggtggaggccgtggaggaagctgtcaagaatgcagccagctacctttctgatgagctcttccttgatgtaagaagatgggacgaagaagagattttgaaggacgaggagcataaactggaggaagaggagtttagttcttatgattcaaaaagcataactcaatactga

SEQ ID NO: 35 - DNA, 2862 bp, Sorghum sp.
  CDS (1)...(2859)
atggaagctattgtgtgtgcatcacatggagccatgggctccttgctgtggaagctgggtgcattgctctctgatgagtacaagcttctaagtagtgtgaaggtggatatgatgttccttaaagctgagcttgaggtcatgcatgctttcctgaagaagatgtcggaggtggaggttcctgatgagcagtccaagtgctggatgaaggaggtgcgggagctgtcctacgacattgaggacagcatcgacagtttcatattttctcttggttgtgagtccaacagcgaacctagaggcttcaaggggtttgttggtaggtgcttgaacttgtttgccgatgctaagacacgtcattggattgccaagaagatccaacgtctcaaatgccatgttatagaggctagcaatcggcgtgggaggtacagggttgatgatgctgtccccagattgagtagaacaagcatagaccctcgcttgccggcattgtacactgagacaacaaggcttgttggtgttgatggcccaagggacaaacttatcaagttgctaacagaaagagagggcacaacgacacagctgagtgtggtctccattgttggatttggtgggcttgggaagactactcttgccaatgaagtgtaccagaagcttgaagggcaatttgactacagagcttttgtgtcagtgtcacaaaaacctgacattaagaagatattgaggcatatactctgccagtatagttgccgagagtgtggcaacaatgaaatatgggatgagcagcaactcatcaacacaacaaggcagttccttaaggataagcggtactttattgtaattgatgatatatggagcatatcagcatggagaactatcagatgtgcttttcctgaaaataattgttccagtagaatattgacaactactcgcatcatcacagtcgctaagtattgttgctctcctcaccgcgaccatgtttatgaactgaagcctctcgatgcagctcactctaagagcttattttttaatagaatttttggttctgaagatagatgtcctcttcatctgaaagaagtttccaatggaatattgaaaaaatgtggtggcttaccattggcaatcattaccgtagctagtttattggtcactaaagctattacaaaagaagaatgggagaagatgctgaagtctattggttcagcacttgaaaaagatacagatatggaagagatgaagaagattcttttgcttagttacaatgatctcccctaccacttgaagacatgcttgttatatctaggtgtgtttcctgaagattatgagattaagagggatcgactgataagaagatggattgctgaaggttttatcactacagaaggtggacaagacatggaggaaataggagaatgctatttcaatgaacttatcaacaggagcatgattcagcctgttggaattcagtatgatggtcgggctgatgcttgccgtgtccatgatatgattcttgatctcatcatatccaaatcagttgaagaaaactttctaaccttatgtggtgatggaaatcacaagctgttgcaacaggataaggttcgtcggctatccatcaattatcatgctcgagatgatattatagtaccaacaaatatgattgtttctaatgttcgatccctcactatatttgggtatgatgagaatatgcctggtctttccaactttctactcttgcgagttctggatctagaaaatagagtggtgctggaatacaattaccttagacacataggtaggctttctcagttgaggtacctccgactcagttcaagaagaataactgcacttcctgaacaaataggagatctacaaaacttgcagaccttagatctgaggtggacaaggattaaaagattgccacagagtgttgtcctcctacgacgattgacatgcttgttggttaacagtttagaattgcctgaaggaattggaaatatgcaagctctacaggagttatcagagattgaaattaactgtcatacatcagtgtcctctttgctggaactgggcaaattgactaatataagaattcttgggctaaattggtgcatcctcgatacaaattatgtaacaaaaatccatgcagatagtttggttatgtccctctgcaaattaggcatgctcaatcttcgatcgatacagattcaaagctatcatagttgttcccttgatttcttaaaggattcttggtttcctcctcctcgtcgcctccaaatatttgatatgtccatagattaccatttccccagaattccaaactggataatctcactggagtacctcagttacctagacatatatcttactccagtggatgaggaatcatttcgaaccctgggggatttgccctctctgttgttcctctggatatcctcaagagaagcaaagcctaaagaaggggttattgtcagcagcaatggtttccgctgtctgaaggagttctacttcacctgttgggaaattggtacagggctgtcttttgaaccaggagccatgccaatgcttgaaaaactgcggattccattcaacgcgcatggtgtgtgctctttgcacggtgttctggattctggaatctggcacctctgttccctaaggcatctccatgtcgagattatttgtcatggtgcgaggcttaaggaggtggaggccgtggaggaagctgtcaagaatgcagccagctacctttctgatgagctcttccttgatgtaagaagatgggacgaagaagagattttgaaggacgaggagcataaactggaggaagaggagtttagttcttatgattcaaaaagcataactcaatactga

SEQ ID NO: 36 - protein, 953 aa, Sorghum sp.
MEAIVCASHGAMGSLLWKLGALLSDEYKLLSSVKVDMMFLKAELEVMHAFLKKMSEVEVPDEQSKCWMKEVRELSYDIEDSIDSFIFSLGCESNSEPRGFKGFVGRCLNLFADAKTRHWIAKKIQRLKCHVIEASNRRGRYRVDDAVPRLSRTSIDPRLPALYTETTRLVGVDGPRDKLIKLLTEREGTTTQLSVVSIVGFGGLGKTTLANEVYQKLEGQFDYRAFVSVSQKPDIKKILRHILCQYSCRECGNNEIWDEQQLINTTRQFLKDKRYFIVIDDIWSISAWRTIRCAFPENNCSSRILTTTRIITVAKYCCSPHRDHVYELKPLDAAHSKSLFFNRIFGSEDRCPLHLKEVSNGILKKCGGLPLAIITVASLLVTKAITKEEWEKMLKSIGSALEKDTDMEEMKKILLLSYNDLPYHLKTCLLYLGVFPEDYEIKRDRLIRRWIAEGFITTEGGQDMEEIGECYFNELINRSMIQPVGIQYDGRADACRVHDMILDLIISKSVEENFLTLCGDGNHKLLQQDKVRRLSINYHARDDIIVPTNMIVSNVRSLTIFGYDENMPGLSNFLLLRVLDLENRVVLEYNYLRHIGRLSQLRYLRLSSRRITALPEQIGDLQNLQTLDLRWTRIKRLPQSVVLLRRLTCLLVNSLELPEGIGNMQALQELSEIEINCHTSVSSLLELGKLTNIRILGLNWCILDTNYVTKIHADSLVMSLCKLGMLNLRSIQIQSYHSCSLDFLKDSWFPPPRRLQIFDMSIDYHFPRIPNWIISLEYLSYLDIYLTPVDEESFRTLGDLPSLLFLWISSREAKPKEGVIVSSNGFRCLKEFYFTCWEIGTGLSFEPGAMPMLEKLRIPFNAHGVCSLHGVLDSGIWHLCSLRHLHVEIICHGARLKEVEAVEEAVKNAASYLSDELFLDVRRWDEEEILKDEEHKLEEEEFSSYDSKSITQY

SEQ ID NO: 37 - protein, 93 aa, Zea mays
FLLVLDDVWNEDPEKWDRYRCALLSGGKGSRIIITTRNKNVGILMGGMTPYHLKQLSNDDCWQLFKKHAFVDGDSSSHPELEIIGKDIVKKLK

SEQ ID NO: 38 - protein, 93 aa, Zea mays
YFVVIDDIWSTSAWRIIRCAFPENNCSSRILTTTRIVTVAKYCCSPHLDRVYALEPLDAAHSESLFLSRIFGSEDRCPLHLKEASDKILKRCG

SEQ ID NO: 39 - protein, 88 aa, Zea mays
  VARIANT (1)...(88): Xaa = Any Amino Acid
FLLVIDGDVSNSDCXAILAALPEKNNGSRIVRIMQGIRKXPRGIAAKHWIELKCFETEKTTSXFSQRVCMEEKINIENFDEVLHGITK

SEQ ID NO: 40 - protein, 92 aa, Oryza sp.
FFLVLDNVWNVQKEIWDALLSLLVGAQLGMILLTTRDETISKMIGTMPSYDLSFLTSEESWQLFKQMAFGFIDQHMDQQFEGFGRKIVGKCG

SEQ ID NO: 41 - protein, 90 aa, Oryza sp.
  VARIANT (1)...(90): Xaa = Any Amino Acid
FFLVLDDVWHYKAWEDLLRTPLNAAATGIILVTTRDETIARVIGVDRTHRVDLMSADVGWELLWRSMNIKXEKQVKNLRDTGIEIVRKCG

SEQ ID NO: 42 - protein, 93 aa, Oryza sp.
FLLVLDDVWNEDPDRWDRYRRALVAGAKGSKIMVTTRNENVGKLMGGLTPYYLKQLSYNDSWHLFISYAFVDGDSSAHPSLEMIGKEIVHKLK

SEQ ID NO: 43 - protein, 92 aa, Oryza sp.
LFFVLDDVWSECESSWHDLIRPLSYALTVIILVTTRSKEVARLAGTVKPFYLTAIPNDDCWLLFQHFAFGKQCVNEKSSLVQIGKKILQKCG

SEQ ID NO: 44 - protein, 90 aa, Sorghum sp.
FFLVLDDIWQHEVWTNLLRAPLNTAATGTILVTTRNDTVARAIGVEDIHRVELMSDEVGWKLLLKSMNISKESEVENLRVLGVDIVRLCG

SEQ ID NO: 45 - protein, 90 aa, Sorghum sp.
  VARIANT (1)...(90): Xaa = Any Amino Acid
FLLVLDDLWESDVWTNLLRTPLAAADQVTILVTTRHDTVAKAIGVGHMHRVELLSEEVGWELLWKSMXISSEKEVLNLRETGIGIVQKCG

SEQ ID NO: 46 - protein, 95 aa, Sorghum sp.
FLLVLDDMWTDHEDEWKKLLAPFKKMQTKGNMAIVTTRIPKVARMVATVGCQIRLERLSDEECMCFFQECVFGNRQTWEGHAILHDFGYKIVKRL

SEQ ID NO: 47 - protein, 93 aa, Sorghum sp.
  VARIANT (1)...(93): Xaa = Any Amino Acid
YFIVIDDIWSISAWRTIRCAFPENNCSSRILTTTRIITVAKYCCSPXRDHVYELKPLDAAHSKSLFFNRIFGSEDRCPLHLKEVSNGILKKCG

SEQ ID NO: 48 - protein, 85 aa, Sorghum sp.
  VARIANT (1)...(85): Xaa = Any Amino Acid
YLIVIDDIQMDEWRTVKSVFEHSSTSSRIILTTTIQPMANMCSSHGNGYVYQMDTLGEEDSKKIAXPGIRSPELEHGSAALLRKC

SEQ ID NO: 49 - protein, 92 aa, Sorghum sp.
YLIIIDDIWSILAWDAIKYAFPENNFSSRIIATTRIVDVARSCCLGGNDRMYEMEALSGLHSKKLFFKRTFGSEDCCPDVLKEVSNEILKKC

SEQ ID NO: 50 - protein, 91 aa, Linum unitatissimum
ILVVLDDVDEKFKFEDMLGSPKDFISQSRFIITSRSMRVLGTLNENQCKLYEVGSMSKPRSLELFSKHAFKKNTPPSYYETLANDVVDTTA

SEQ ID NO: 51 - protein, 88 aa, Nicotiana glutinosa
VLIVLDDIDNKDHYLEYLAGDLDWFGNGSRIIITTRDKHLIEKNDIIYEVTALPDHESIQLFKQHAFGKEVPNENFEKLSLEVVNYAK

SEQ ID NO: 52 - protein, 90 aa, Lycopersicon esculentum
FLILIDDVWDYKVWDNLCMCFSDVSNRSRIILTTRLNDVAEYVKCESDPHHLRLFRDDESWTLLQKEVFQGESCPPELEDVGFEISKSCR

SEQ ID NO: 53 - protein, 90 aa, Arabidopsis thaliana
FLLLLDDVWEEIDLEKTGVPRPDRENKCKVMFTTRSIALCNNMGAEYKLRVEFLEKKHAWELFCSKVWRKDLLESSSIRRLAEIIVSKCG

SEQ ID NO: 54 - protein, 94 aa, Arabidopsis thaliana
YIVVLDDVWTTGLWREISIALPDGIYGSRVMMTTRDMNVASFPYGIGSTKHEIELLKEDEAWVLFSNKAFPASLEQCRTQNLEPIARKLVERCQ

(19) United States
(12) Patent Application Publication
(10) Pub. No.: US 2003/0088887 A1
(43) Pub. Date: May 8, 2003

(54) Compositions and methods for enhancing disease resistance in plants

(75) Inventor: Jeffrey L. Bennetzen, West Lafayette, IN (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Purdue Research Foundation

(21) Appl. No.: 10/267,718

(22) Filed: Oct. 9, 2002

Related U.S. Application Data
(63) Continuation of application No. 09/354,453, filed on Jul. 15, 1999, now abandoned.
(60) Provisional application No. 60/093,290, filed on Jul. 17, 1998.

(51) Int. Cl. A01H 1/00; C12N 9/24; C07H 21/04; C12N 15/82; C12N 5/04

(52) U.S. Cl. 800/279; 435/200; 435/419; 435/69.1; 435/320.1; 536/23.2

(57) ABSTRACT

Compositions and methods for enhancing or creating plant disease resistance to plant pests are provided. Transforming a plant with a novel maize, sorghum, or rice disease resistance gene homologue (RGH) of the invention enhances disease resistance of the plant. Transformed plants, plant cells, tissues, and seed having enhanced disease resistance are also provided.

### CROSS-REFERENCE TO RELATED APPLICATIONS

This application is a continuation of U.S. application Ser. No. 09/354,453, filed Jul. 15, 1999 and also claims the benefit of U.S. Provisional Application Serial No. 60/093,290, filed Jul. 17, 1998, each of which are hereby incorporated in their entirety by reference herein.

### FIELD OF THE INVENTION

The invention relates to the genetic manipulation of plants, particularly to transforming plants with genes that enhance disease resistance.

### BACKGROUND OF THE INVENTION

Disease in plants is caused by biotic and abiotic causes. Biotic causes include fungi, viruses, bacteria, and nematodes. Of these, fungi are the most frequent causative agent of disease in plants. Abiotic causes of disease in plants include extremes of temperature, water, oxygen, soil pH, plus nutrient-element deficiencies and imbalances, excess heavy metals, and air pollution.

A host of cellular processes enables plants to defend themselves from disease caused by pathogenic agents. These processes apparently form an integrated set of resistance mechanisms that is activated by initial infection and then limits further spread of the invading pathogenic microorganism. Subsequent to recognition of a potentially pathogenic microbe, plants can activate an array of biochemical responses. Generally, the plant responds by inducing several local responses in the cells immediately surrounding the infection site. The most common resistance response observed in both nonhost and race-specific interactions is termed the “hypersensitive response” (HR). In the hypersensitive response, cells contacted by the pathogen, and often neighboring cells, rapidly collapse and dry in a necrotic fleck. Other responses include the deposition of callose, the physical thickening of cell walls by lignification, and the synthesis of various antibiotic small molecules and proteins. Genetic factors in both the host and the pathogen determine the specificity of these local responses, which can be very effective in limiting the spread of infection.

The hypersensitive response in many plant-pathogen interactions results from the expression of a resistance (R) gene in the plant and a corresponding avirulence (avr) gene in the pathogen. This interaction is associated with the rapid, localized cell death of the hypersensitive response. R genes that respond to specific bacterial, fungal, or viral pathogens, have been isolated from a variety of plant species and several appear to encode cytoplasmic proteins.

The resistance gene in the plant and the avirulence gene in the pathogen often conform to a gene-for-gene relationship. That is, resistance to a pathogen is only observed when the pathogen carries a specific avirulence gene and the plant carries a corresponding or complementing resistance gene. Because avrR gene-for-gene relationships are observed in many plant-pathogen systems and are accompanied by a characteristic set of defense responses, a common molecular mechanism underlying avrR gene mediated resistance has been postulated. A simple model which has been proposed is that pathogen avr genes directly or indirectly generate a specific molecular signal (ligand) that is recognized by cognate receptors encoded by plant R genes. Recently, direct evidence for a physical interaction between paired R and avr gene products as receptors and ligands in plant-pathogen recognition is provided by an interaction between tomato Pto and Pseudomonas syringe avrPto products (Scofield et al. (1996) Science 274:2063-2065; Tang et al. (1996) Science 274:2060-2063.

Both plant resistance genes and corresponding pathogen avirulence genes have been cloned. The plant kingdom contains thousands of R genes with specific specificities for viral, bacterial, fungal, or nematode pathogens. Although there are differences in the defense responses induced during different plant-pathogen interactions, some common themes are apparent among R gene-mediated defenses. The function of a given R gene is dependent on the genotype of the pathogen. Plant pathogens produce a diversity of potential signals, and in a fashion analogous to the production of antigens by mammalian pathogens, some of these signals are detectable by some plants.

The avirulence gene causes the pathogen to produce a signal that triggers a strong defense response in a plant with the appropriate R gene. However, expressing an avirulence gene does not stop the pathogen from being virulent on hosts that lack the corresponding R gene. A single plant can have many R genes, and a pathogen can have many avr genes.

As noted, among the causative agents of infectious disease of crop plants, the phytopathogenic fungi play the dominant role. Phytopathogenic fungi cause devastating epidemics, as well as causing significant annual crop yield losses. All of the approximately 300,000 species of flowering plants are attacked by pathogenic fungi. However, a single plant species can be host to only a few fungal species, and similarly, most fungi usually have a limited host range.

Plant disease outbreaks have resulted in catastrophic crop failures that have triggered famines and caused major social change. Generally, the best strategy for plant disease control is to use resistant cultivars selected or developed by plant breeders for this purpose. However, the potential for serious crop disease epidemics persists today, as evidenced by outbreaks of the Victoria blight of oats and southern corn leaf blight. Accordingly, molecular methods are needed to supplement traditional breeding methods to protect plants from pathogen attack.

### SUMMARY OF THE INVENTION

Compositions and methods for creating or enhancing resistance to plant pests are provided. Compositions are nucleotide sequences for novel disease resistance gene homologues cloned from maize, rice, and sorghum and the amino acid sequences for the proteins or partial-length proteins or polypeptides encoded thereby. Methods of the invention involve stably transforming a plant with one of these novel disease resistance gene homologues operably linked with a promoter capable of driving expression of a nucleotide coding sequence in a plant cell. Expression of the novel nucleotide sequences confers disease resistance to a plant by interacting with the complementing phytopathogen avirulence gene product released into the plant by the invading plant pathogen. The methods of the invention find use in controlling plant pests, including fungal pathogens, viruses, nematodes, insects, and the like.

Transformed plants and seeds, as well as methods for making such plants and seeds are additionally provided.

### BRIEF DESCRIPTION OF THE DRAWINGS

FIGS. 1A-1B shows an alignment of conserved regions of the deduced amino acid sequences encoded by the maize, rice, and sorghum resistance gene homologues (RGHs) of the invention with several other R genes. The alignment starts with the third amino acid residue within the kinase-2 domain, a sequence feature shared by disease resistance proteins encoded by R genes in the NBS-LRR superfamily. Three of the novel sequences shown in the alignment are from maize (M05, also referred to as M5-1, SEQ ID NO: 37; M06, also referred to as M6-1, SEQ ID NO: 38; Mr05, also referred to as M5-6, SEQ ID NO: 39), four are from rice (R0501, also referred to as R5-1, SEQ ID NO: 40; R0502, also referred to as R5-2, SEQ ID NO: 41; R0503, also referred to as R5-3, SEQ ID NO: 42; R0518, also referred to as R5-4, SEQ ID NO: 43), and six are from sorghum (S0510, also referred to as S5-5, SEQ ID NO: 44; S0535, also referred to as S5-2A, SEQ ID NO: 45; S0545, also referred to as S5-2B, SEQ ID NO: 46; S0606, also referred to as S6-1, SEQ ID NO: 47; S0608, also referred to as S6-2, SEQ ID NO: 48; and S11-1, SEQ ID NO: 49). These sequences are aligned with the corresponding conserved regions of flax L6 (SEQ ID NO: 50), tobacco N (SEQ ID NO: 51), tomato Prf (SEQ ID NO: 52), and Arabidopsis RPS2 (SEQ ID NO: 53) and RPM1 (SEQ ID NO: 54). The alignment was generated using PRETTYBOX function of GCG sequence analysis packages. SEQ ID NOs shown in parentheses set forth that portion of a particular RGH polypeptide of the invention that is shown in this figure.

FIG. 2 shows an alignment of kinase-2 domains of the novel RGHs M6-1, S6-1, S6-2, and S11-1 with the kinase-2 domains of tomato Prf, and Arabidopsis RPS2 and RPM1. Note that the putative introns have been removed from the deduced amino acid sequences of the novel RGHs and are shown as asterisks.

FIG. 3 shows sequence features of the S6-1 gene (SEQ ID NO: 34) subcloned from a sorghum BAC clone. Black and hatched boxes represent coding regions and open boxes represent the putative intron located in the kinase-2 domain. The nucleotide numbers are shown above boxes and in italic, and numbers of the deduced amino acids are shown below boxes. LZ, leucine zipper; P, P-loop; K2, kinase-2; K3a, kinase-3a; TM, a putative transmembrane domain; X, conserved domain X; Y, conserved domain Y; LRRs, leucine-rich repeats. The similarity of each domain in S6-1 to the corresponding region of Arabidopsis RPM1 is indicated below individual domains. The corresponding PCR RGH clone of S6-1 (PCR S6-1; SEQ ID NO: 21) is also shown.

FIG. 4 schematically shows a plasmid vector comprising a RGH sequence of the invention operably linked to the ubiquitin promoter.

### DETAILED DESCRIPTION OF THE INVENTION

The invention is drawn to compositions and methods for creating or enhancing resistance in a plant to plant pests. Accordingly, the compositions and methods are also useful in protecting plants against fungal pathogens, viruses, nematodes, insects, and the like.

By “disease resistance” is intended that the plants avoid the disease symptoms that are the outcome of plant-pathogen interactions. That is, pathogens are prevented from causing plant diseases and the associated disease symptoms. The compositions and methods of the invention can be utilized to protect plants from disease, particularly those diseases that are caused by plant pathogens.

Pathogens of the invention include, but are not limited to, viruses or viroids, bacteria, insects, nematodes, fungi, and the like. Viruses include any plant virus, for example, tobacco or cucumber mosaic virus, ringspot virus, necrosis virus, maize dwarf mosaic virus, etc. Specific fungal and viral pathogens for the major crops include:

Soybeans: Phytophthora megasperma fsp. glycinea, Macrophomina phaseolina, Rhizoctonia solani, Sclerotinia sclerotiorum, Fusarium oxysporum, Diaporthe phaseolorum var. sojae (Phomopsis sojae), Diaporthe phaseolorum var. caulivora, Sclerotium rolfsii, Cercospora kikuchii, Cercospora sojina, Peronospora manshurica, Colletotrichum dematium (Colletotichum truncatum), Corynespora cassiicola, Septoria glycines, Phyllosticta sojicola, Alternaria alternata, Pseudomonas syringae p.v. glycinea, Xanthomonas campestris p.v. phaseoli, Microsphaera diffusa, Fusarium semitectum, Phialophora gregata, Soybean mosaic virus, Glomerella glycines, Tobacco Ring spot virus, Tobacco Streak virus, Phakopsora pachyrhizi, Pythium aphanidermatum, Pythium ultimum, Pythium debaryanum, Tomato spotted wilt virus, Heterodera glycines Fusarium solani; Canola: Albugo candida, Alternaria brassicae, Leptosphaeria maculans, Rhizoctonia solani, Sclerotinia sclerotiorum, Mycosphaerella brassiccola, Pythium ultimum, Peronospora parasitica, Fusarium roseum, Alternaria alternata; Alfalfa: Clavibater michiganese subsp. insidiosum, Pythium ultimum, Pythium irregulare, Pythium splendens, Pythium debaryanum, Pythium aphanidermatum, Phytophthora megasperma, Peronospora trifoliorum, Phoma medicaginis var. medicaginis, Cercospora medicaginis, Pseudopeziza medicaginis, Leptotrochila medicaginis, Fusarium, Xanthomonas campestris p.v. alfalfae, Aphanomyces euteiches, Stemphylium herbarum, Stemphylium alfalfae; Wheat: Pseudomonas syringae p.v. atrofaciens, Urocystis agropyri, Xanthomonas campestris p.v. translucens, Pseudomonas syringae p.v. syringae, Alternaria alternata, Cladosporium herbarum, Fusarium graminearum, Fusarium avenaceum, Fusarium culmorum, Ustilago tritici, Ascochyta tritici, Cephalosporium gramineum, Collotetrichum graminicola, Erysiphe graminis f.sp. tritici, Puccinia graminis f.sp. tritici, Puccinia recondita f.sp. tritici, Puccinia striiformis, Pyrenophora tritici-repentis, Septoria nodorum, Septoria tritici, Septoria avenae, Pseudocercosporella herpotrichoides, Rhizoctonia solani, Rhizoctonia cerealis, Gaeumannomyces graminis var. tritici, Pythium aphanidermatum, Pythium arrhenomanes, Pythium ultimum, Bipolaris sorokiniana, Barley Yellow Dwarf Virus, Brome Mosaic Virus, Soil Borne Wheat Mosaic Virus, Wheat Streak Mosaic Virus, Wheat Spindle Streak Virus, American Wheat Striate Virus, Claviceps purpurea, Tilletia tritici, Tilletia laevis, Ustilago tritici, Tilletia indica, Rhizoctonia solani, Pythium arrhenomannes, Pythium gramicola, Pythium aphanidermatum, High Plains Virus, European wheat striate virus; Sunflower: Plasmophora halstedii, Sclerotinia sclerotiorum, Aster Yellows, Septoria helianthi, Phomopsis helianthi, Alternaria helianthi, Alternaria zinniae, Botrytis cinerea, Phoma macdonaldii, Macrophomina phaseolina, Erysiphe cichoracearum, Rhizopus oryzae, Rhizopus arrhizus, Rhizopus stolonifer, Puccinia helianthi, Verticillium dahliae, Erwinia carotovorum pv. carotovora, Cephalosporium acremonium, Phytophthora cryptogea, Albugo tragopogonis; Corn: Fusarium moniliforme var. subglutinans, Erwinia stewartii, Fusarium moniliforme, Gibberella zeae (Fusarium graminearum), Stenocarpella maydi (Diplodia maydis), Pythium irregulare, Pythium debaryanum, Pythium graminicola, Pythium splendens, Pythium ultimum, Pythium aphanidermatum, Aspergillusflavus, Bipolaris maydis O, T (Cochliobolus heterostrophus), Helminthosporium carbonum I, II & III (Cochliobolus carbonum), Exserohilum turcicum I, II & III, Helminthosporium pedicellatum, Physoderma maydis, Phyllosticta maydis, Kabatiella-maydis, Cercospora sorghi, Ustilago maydis, Puccinia sorghi, Puccinia polysora, Macrophomina phaseolina, Penicillium oxalicum, Nigrospora oryzae, Cladosporium herbarum, Curvularia lunata, Curvularia inaequalis, Curvularia pallescens, Clavibacter michiganense subsp. nebraskense, Trichoderma viride, Maize Dwarf Mosaic Virus A & B, Wheat Streak Mosaic Virus, Maize Chlorotic Dwarf Virus, Claviceps sorghi, Pseudonomas avenae, Erwinia chrysanthemi pv. zea, Erwinia carotovora, Corn stunt spiroplasma, Diplodia macrospora, Sclerophthora macrospora, Peronosclerospora sorghi, Peronosclerospora philippinensis, Peronosclerospora maydis, Peronosclerospora sacchari, Sphacelotheca reiliana, Physopella zeae, Cephalosporium maydis, Cephalosporium acremonium, Maize Chlorotic Mottle Virus, High Plains Virus, Maize Mosaic Virus, Maize Rayado Fino Virus, Maize Streak Virus, Maize Stripe Virus, Maize Rough Dwarf Virus; Sorghum: Exserohilum turcicum, Colletotrichum graminicola (Glomerella graminicola), Cercospora sorghi, Gloeocercospora sorghi, Ascochyta sorghina, Pseudomonas syringae p.v. syringae, Xanthomonas campestris p.v. holcicola, Pseudomonas andropogonis, Puccinia purpurea, Macrophomina phaseolina, Perconia circinata, Fusarium moniliforme, Alternaria alternata, Bipolaris sorghicola, Helminthosporium sorghicola, Curvularia lunata, Phoma insidiosa, Pseudomonas avenae (Pseudomonas alboprecipitans), Ramulispora sorghi, Ramulispora sorghicola, Phyllachara sacchari, Sporisorium reilianum (Sphacelotheca reiliana), Sphacelotheca cruenta, Sporisorium sorghi, Sugarcane mosaic H, Maize Dwarf Mosaic Virus A & B, Claviceps sorghi, Rhizoctonia solani, Acremonium strictum, Sclerophthona macrospora, Peronosclerospora sorghi, Peronosclerospora philippinensis, Sclerospora graminicola, Fusarium graminearum, Fusarium oxysporum, Pythium arrhenomanes, Pythium graminicola, etc.

Nematodes include parasitic nematodes such as root-knot, cyst, lesion, and renniform nematodes, etc.

Insect pests include insects selected from the orders Coleoptera, Diptera, Hymenoptera, Lepidoptera, Mallophaga, Homoptera, Hemiptera, Orthoptera, Thysanoptera, Dermaptera, Isoptera, Anoplura, Siphonaptera, Trichoptera, etc., particularly Coleoptera and Lepidoptera. Insect pests of the invention for the major crops include: Maize: Ostrinia nubilalis, European corn borer; Agrotis ipsilon, black cutworm; Helicoverpa zea, corn earworm; Spodoptera frugiperda, fall armyworm; Diatraea grandiosella, southwestern corn borer; Elasmopalpus lignosellus, lesser cornstalk borer; Diatraea saccharalis, surgarcane borer; Diabrotica virgifera, western corn rootworm; Diabrotica longicornis barberi, northern corn rootworm; Diabrotica undecimpunctata howardi, southern corn rootworm; Melanotus spp., wireworms; Cyclocephala borealis, northern masked chafer (white grub); Cyclocephala immaculata, southern masked chafer (white grub); Popillia japonica, Japanese beetle; Chaetocnema pulicaria, corn flea beetle; Sphenophorus maidis, maize billbug; Rhopalosiphum maidis, corn leaf aphid; Anuraphis maidiradicis, corn root aphid; Blissus leucopterus leucopterus, chinch bug; Melanoplusfemurrubrum, redlegged grasshopper; Melanoplus sanguinipes, migratory grasshopper; Hylemya platura, seedcorn maggot; Agromyza parvicornis, corn blot leafminer; Anaphothrips obscrurus, grass thrips; Solenopsis milesta, thief ant; Tetranychus urticae, twospotted spider mite; Sorghum: Chilo partellus, sorghum borer; Spodopterafrugiperda, fall armyworm; Helicoverpa zea, corn earworm; Elasmopalpus lignosellus, lesser cornstalk borer; Feltia subterranea, granulate cutworm; Phyllophaga crinita, white grub; Eleodes, Conoderus, and Aeolus spp., wireworms; Oulema melanopus, cereal leaf beetle; Chaetocnema pulicaria, corn flea beetle; Sphenophorus maidis, maize billbug; Rhopalosiphum maidis; corn leaf aphid; Sipha flava, yellow sugarcane aphid; Blissus leucopterus leucopterus, chinch bug; Contarinia sorghicola, sorghum midge; Tetranychus cinnabarinus, carmine spider mite; Tetranychus urticae, twospotted spider mite; Wheat: Pseudaletia unipunctata, army worm; Spodoptera frugiperda, fall armyworm; Elasmopalpus lignosellus, lesser cornstalk borer; Agrotis orthogonia, western cutworm; Elasmopalpus lignosellus, lesser cornstalk borer; Oulema melanopus, cereal leaf beetle; Hypera punctata, clover leaf weevil; Diabrotica undecimpunctata howardi, southern corn rootworm; Russian wheat aphid; Schizaphis graminum, greenbug; Macrosiphum avenae, English grain aphid; Melanoplus femurrubrum, redlegged grasshopper; Melanoplus differentialis, differential grasshopper; Melanoplus sanguinipes, migratory grasshopper; Mayetiola destructor, Hessian fly; Sitodiplosis mosellana, wheat midge; Meromyza americana, wheat stem maggot; Hylemya coarctata, wheat bulb fly; Frankliniella fusca, tobacco thrips; Cephus cinctus, wheat stem sawfly; Aceria tulipae, wheat curl mite; Sunflower: Suleima helianthana, sunflower bud moth; Homoeosoma electellum, sunflower moth; zygogramma exclamationis, sunflower beetle; Bothyrus gibbosus, carrot beetle; Neolasioptera murtfeldtiana, sunflower seed midge; Cotton: Heliothis virescens, cotton budworm; Helicoverpa zea, cotton bollworm; Spodoptera exigua, beet armyworm; Pectinophora gossypiella, pink bollworm; Anthonomus grandis grandis, boll weevil; Aphis gossypii, cotton aphid; Pseudatomoscelis seriatus, cotton fleahopper; Trialeurodes abutilonea, bandedwinged whitefly; Lygus lineolaris, tarnished plant bug; Melanoplusfemurrubrum, redlegged grasshopper; Melanoplus differentialis, differential grasshopper; Thrips tabaci, onion thrips; Franklinkiella fusca, tobacco thrips; Tetranychus cinnabarinus, carmine spider mite; Tetranychus urticae, twospotted spider mite; Rice: Diatraea saccharalis, sugarcane borer; Spodoptera frugiperda, fall armyworm; Helicoverpa zea, corn earworm; Colaspis brunnea, grape colaspis; Lissorhoptrus oryzophilus, rice water weevil; Sitophilus oryzae, rice weevil; Nephotettix nigropictus, rice leafhopper; Blissus leucopterus leucopterus, chinch bug; Acrosternum hilare, green stink bug; Soybean: Pseudoplusia includens, soybean looper; Anticarsia gemmatalis, velvetbean caterpillar; Plathypena scabra, green cloverworm; Ostrinia nubilalis, European corn borer; Agrotis ipsilon, black cutworm; Spodoptera exigua, beet armyworm; Heliothis virescens, cotton budworm; Helicoverpa zea, cotton bollworm; Epilachna varivestis, Mexican bean beetle; Myzus persicae, green peach aphid; Empoasca fabae, potato leafhopper; Acrosternum hilare, green stink bug; Melanoplus femurrubrum, redlegged grasshopper; Melanoplus differentialis, differential grasshopper; Hylemya platura, seedcorn maggot; Sericothrips variabilis, soybean thrips; Thrips tabaci, onion thrips; Tetranychus turkestani, strawberry spider mite; Tetranychus urticae, twospotted spider mite; Barley: Ostrinia nubilalis, European corn borer; Agrotis ipsilon, black cutworm; Schizaphis graminum, greenbug; Blissus leucopterus leucopterus, chinch bug; Acrosternum hilare, green stink bug; Euschistus servus, brown stink bug; Delia platura, seedcorn maggot; Mayetiola destructor, Hessian fly; Petrobia latens, brown wheat mite; Oil Seed Rape: Brevicoryne brassicae, cabbage aphid; Phyllotreta cruciferae, Flea beetle; Mamestra configurata, Bertha armyworm; Plutella xylostella, Diamond-back moth; Delia ssp., Root maggots.

Compositions of the invention include resistance gene homologues (RGHs) that are involved in plant disease resistance. In particular, the present invention provides for isolated nucleic acid molecules comprising nucleotide sequences encoding the amino acid sequences shown in SEQ ID NOS: 2, 5, 7, 9, 11, 13, 15, 17, 19, 23, 26, 29, and 36. Further provided are polypeptides having an amino acid sequence encoded by a nucleic acid molecule described herein, for example those set forth in SEQ ID NOS: 1, 3, 4, 6, 8, 10, 12, 14, 16, 18, 20, 21, 22, 24, 25, 27, 28, 30, 31, 32, 34, and 35, and fragments and variants thereof.

The naturally occurring disease resistance proteins or partial-length proteins encoded by the disclosed RGH nucleotide sequences, and fragments and variants thereof, are encompassed by the present invention. Where putative introns occur within the disclosed nucleotide sequences (such as in SEQ ID NOS: 3, 21, 24, 27, 30, 31, 32, and 34), compositions of the invention also encompass the mature form of the protein or partial-length protein encoded thereby following intron removal.

Compositions of the invention include isolated nucleic acid molecules comprising novel RGH sequences isolated from maize, rice, and sorghum. The RGH sequences isolated from maize are partial gene sequences designated as clones M5-1 (SEQ ID NO: 1), M6-1 (SEQ ID NO: 3, which sets forth the M6-1 sequence with its putative 126-bp intron, and SEQ ID NO: 4, which sets forth the M6-1 sequence with the putative intron removed), and M5-6 (SEQ ID NO: 6). These maize RGHs are partial open reading frames encoding polypeptides having the predicted amino acid sequences set forth in SEQ ID NOS: 2, 5, and 7, respectively.

The RGH sequences isolated from rice are partial gene sequences designated as clones R5-1 (SEQ ID NO: 8), R5-2 (SEQ ID NO: 10), R5-3 (SEQ ID NO: 12), and R5-4 (SEQ ID NO: 14). These RGHs are partial open reading frames encoding polypeptides having the predicted amino acid sequences set forth in SEQ ID NOS: 9, 11, 13, and 15, respectively.

The RGH sequences isolated from sorghum are partial gene sequences designated as clones S5-5 (SEQ ID NO: 16), S5-2A (SEQ ID NO: 18), S5-2B (SEQ ID NO: 20), S6-1 (SEQ ID NO: 21, which sets forth the S6-1 sequence with its putative 92-bp intron, and SEQ ID NO: 22, which sets forth the S6-1 sequence with the putative intron removed); S6-2 (SEQ ID NO: 24, which sets forth the S6-2 sequence with its putative 100-bp intron, and SEQ ID NO: 25, which sets forth the S6-2 sequence with its putative intron removed); S11-1 (SEQ ID NO: 27, which sets forth the S11-1 sequence with its putative 518-bp intron, and SEQ ID NO: 28, which sets forth the S11-1 sequence with its putative intron removed); S11-25 (SEQ ID NO: 30, which sets forth the S11-25 sequence without removal of a putative intron); S11-27 (SEQ ID NO: 31, which sets forth the S11-27 sequence without removal of a putative intron); and S11-34 (SEQ ID NO: 32, which sets forth the S11-34 sequence without removal of a putative intron). The full-length open reading frame sequence for the clone designated S6-1 and referred to as the S6-1 gene is also provided. The full-length open reading frame for the S6-1 gene is set forth as SEQ ID NO: 34 (which includes the putative 92-bp intron) and SEQ ID NO: 35 (which shows the S6-1 sequence with the putative intron removed).

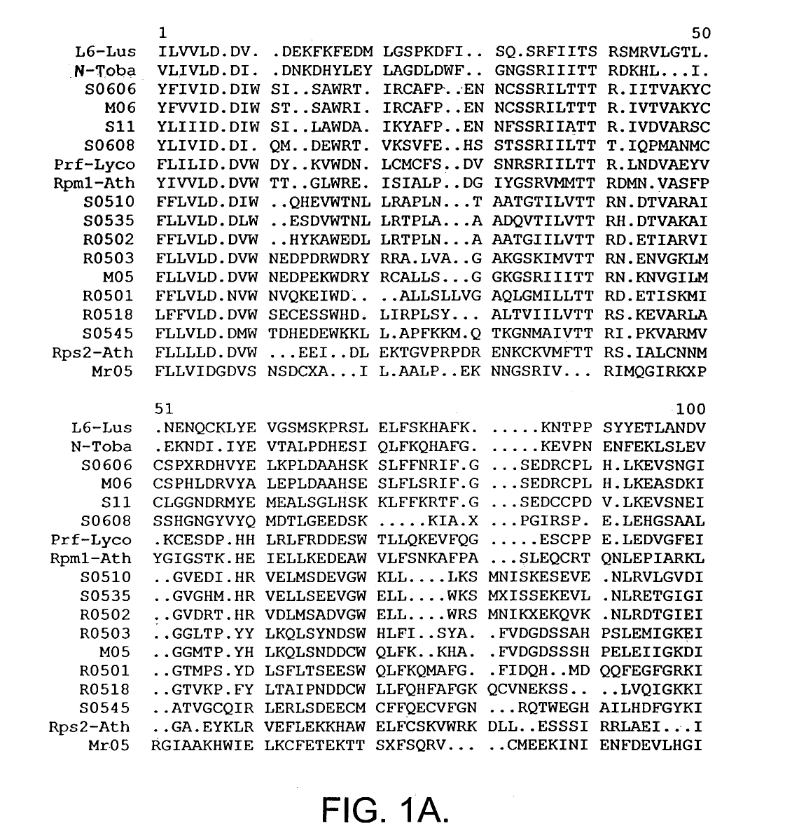

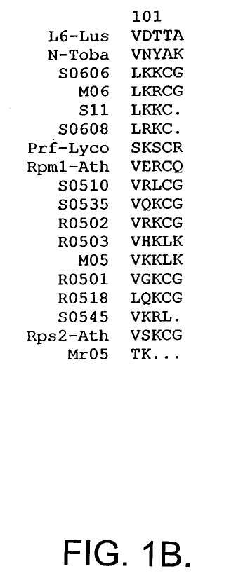

The sorghum clones designated S5-5, S5-2A, S6-1, S6-2, and S11-1 encode polypeptides having the predicted amino acid sequences set forth in SEQ ID NOS: 17, 19, 23, 26, and 29, respectively. The sorghum clone designated S5-2B encodes a polypeptide that comprises the amino acid sequence set forth in SEQ ID NO: 46, which represents that portion of the polypeptide comprising a kinase-2 domain characteristic of products of R genes in the NBS-LRR superfamily (see FIGS. 1A-1B, and the sequence referred to as S0545). The full-length open reading frame of the S6-1 gene encodes a protein having a predicted amino acid sequence set forth in SEQ ID NO: 36.

The nucleotide sequences of the invention and the amino acid sequences encoded thereby, as well as fragments and variants thereof, are hereinafter referred to as RGH nucleotide sequences and RGH proteins, respectively. Thus, the term RGH protein encompasses the disclosed full-length and partial-length proteins encoded by the RGH nucleotide sequences disclosed herein.

The invention encompasses isolated or substantially purified nucleic acid or protein compositions. An “isolated” or “purified” nucleic acid molecule or protein, or biologically active portion thereof, is substantially free of other cellular material, or culture medium when produced by recombinant techniques, or substantially free of chemical precursors or other chemicals when chemically synthesized. Preferably, an “isolated” nucleic acid is free of sequences (preferably protein encoding sequences) that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. A protein that is substantially free of cellular material includes preparations of protein or polypeptide having less than about 30%, 20%, 10%, 5%, (by dry weight) of contaminating protein. When the protein of the invention, or biologically active portion thereof, is recombinantly produced, preferably culture medium represents less than about 30%, 20%, 10%, or 5% (by dry weight) of chemical precursors or non-protein-of-interest chemicals.

Fragments and variants of the disclosed nucleotide sequences and proteins or partial-length proteins encoded thereby are also encompassed by the present invention. By “fragment” is intended a portion of the nucleotide sequence or a portion of the amino acid sequence, and hence a portion of the polypeptide or protein, encoded thereby. Fragments of a nucleotide sequence may encode protein fragments that retain the biological activity of the native RGH and hence confer disease resistance to a plant by interacting with the complementing phytopathogen avirulence gene product released into the plant by the invading plant pathogen. Alternatively, fragments of a nucleotide sequence that are useful as hybridization probes generally do not encode fragment proteins retaining biological activity. Thus, fragments of a nucleotide sequence may range from at least about 20 nucleotides, about 50 nucleotides, about 100 nucleotides, and up to the full-length nucleotide sequence encoding the RGH proteins of the invention.

A fragment of an RGH nucleotide sequence that encodes a biologically active portion of an RGH protein of the invention will encode at least 15, 20, 25, 30, 50, 100, 150, 200, or 250 contiguous amino acids, or up to the total number of amino acids present in a full-length RGH protein of the invention. Fragments of an RGH nucleotide sequence that are useful as hybridization probes for PCR primers generally need not encode a biologically active portion of an RGH protein.

A fragment of an RGH nucleotide sequence may encode a biologically active portion of an RGH protein, or it may be a fragment that can be used as a hybridization probe or PCR primer using methods disclosed below. A biologically active portion of an RGH protein can be prepared by isolating a portion of one of the RGH nucleotide sequences of the invention, expressing the encoded portion of the RGH protein (e.g., by recombinant expression in vitro), and assessing the activity of the encoded portion of the RGH protein. Nucleic acid molecules that are fragments of an RGH nucleotide sequence comprise at least 16, 20, 50, 75, 100, 150, 200, 250, 300, 350, 400, 450, 500, 600, 700, 800, 900, 1000, 1250, 1500, 1750, 2000, 2250, 2500, 2750, 2800, 2850, 2900, or 2950 nucleotides, or up to the number of nucleotides present in a full-length RGH nucleotide sequence disclosed herein (for example, 517, 634, 508, 498, 515, 506, 518, 510, 506, 505, 514, 609, 517, 605, 505, 1040, 522, 1044, 1038, 1043, 2954, or 2862 nucleotides for SEQ ID NOS: 1, 3, 4, 6, 8, 10, 12, 14, 16, 18, 20, 21, 22, 24, 25, 27, 28, 30, 31, 32, 34, and 35, respectively).

By “variants” is intended substantially similar sequences. For nucleotide sequences, conservative variants include those sequences that, because of the degeneracy of the genetic code, encode the amino acid sequence of one of the disease resistance polypeptides of the invention. Naturally occurring allelic variants such as these can be identified with the use of well-known molecular biology techniques, as, for example, with polymerase chain reaction (PCR) and hybridization techniques as outlined below. Variant nucleotide sequences also include synthetically derived nucleotide sequences, such as those generated, for example, by using site-directed mutagenesis but which still encode an RGH protein of the invention. Generally, nucleotide sequence variants of the invention will have at least 40, 50, 60, to 70%, generally, 80%, preferably 85%, 90%, up to 95%, 98% sequence identity to the native nucleotide sequence.

By “variant” protein is intended a protein derived from the native protein by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Such variants may result from, for example, genetic polymorphism or from human manipulation. Methods for such manipulations are generally known in the art.

The proteins of the invention may be altered in various ways including amino acid substitutions, deletions, truncations, and insertions. Methods for such manipulations are generally known in the art. For example, amino acid sequence variants of the RGH proteins can be prepared by mutations in the DNA. Methods for mutagenesis and nucleotide sequence alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D.C.), herein incorporated by reference. Conservative substitutions, such as exchanging one amino acid with another having similar properties, may be preferred.

Thus, the genes and nucleotide sequences of the invention include both the naturally occurring sequences as well as mutant forms. Likewise, the proteins of the invention encompass both naturally occurring proteins as well as variations and modified forms thereof. Such variants will continue to possess the desired disease resistance activity. Obviously, the mutations that will be made in the DNA encoding the variant must not place the sequence out of reading frame and preferably will not create complementary regions that could produce secondary mRNA structure. See, EP Patent Application Publication No. 75,444.

The deletions, insertions, and substitutions of the protein sequence encompassed herein are not expected to produce radical changes in the characteristics of the protein. However, when it is difficult to predict the exact effect of the substitution, deletion, or insertion in advance of doing so, one skilled in the art will appreciate that the effect will be evaluated by routine screening assays. That is, the activity can be evaluated by monitoring for enhanced disease resistance.

The resistance gene homologues of the invention can be optimized for enhanced expression in plants of interest. See, for example, EPA0359472; WO91/16432; Perlak et al. (1991) Proc. Natl. Acad. Sci USA 88:3324-3328; and Murray et al. (1989) Nucleic Acids Res. 17:477-498. In this manner, the genes or gene fragments can be synthesized utilizing plant-preferred codons. See, for example, Murray et al. (1989) Nucleic Acids Res. 17:477-498, the disclosure of which is incorporated herein by reference. In this manner, synthetic genes can also be made based on the distribution of codons a particular host uses for a particular amino acid. See, for example, Campbell and Gowri (1990) Plant Physiol. 92:1 -11 for a discussion of host-preferred codon usage. Thus, the nucleotide sequences can be optimized for expression in any plant. It is recognized that all or any part of the gene sequence may

be optimized or synthetic. That is, synthetic or partially optimized sequences may also be used.

Variant nucleotide sequences and proteins also encompass sequences and proteins derived from a mutagenic and recombinogenic procedure such as DNA shuffling. With such a procedure, one or more different coding sequences can be manipulated to create a new protein possessing the desired properties. In this manner, libraries of recombinant polynucleotides are generated from a population of related sequence polynucleotides comprising sequence regions that have substantial sequence identity and can be homologously recombined in vitro or in vivo. For example, using this approach, sequence motifs encoding a domain of interest may be shuffled between the resistance gene homologues of the invention and other known disease resistance genes to obtain a new gene coding for a disease resistance protein with an improved property of interest, such as an improved interaction with its complementing phytopathogen avirulence gene product, which in turn enhances disease resistance. Strategies for such DNA shuffling are known in the art. See, for example, Stemmer (1994) Proc. Natl. Acad. Sci. USA 91:10747-10751; Stemmer (1994) Nature 370:389-391; Crameri et al. (1997) Nature Biotech. 15:436-438; Moore et al. (1997) J. Mol. Biol. 272:336-347; Zhang et al. (1997) Proc. Natl. Acad. Sci. USA 94:4504-4509; Crameri et al. (1998) Nature 391:288-291; and U.S. Pat. Nos. 5,605,793 and 5,837,458.

Other resistance genes well known in the art may be used in such a DNA shuffling approach. See, for example, Dixon et al. (1996) Cell 84(3):451-459; Reuber et al. (1996) Plant Cell 8(2):241-249; Grant et al. (1995) Science 269(5225):843-846; Bisgrove et al. (1994) Plant Cell 6(7):927-933; Dangl et al. (1992) Plant Cell 4(11):1359-1369; Ashfield et al. (1995) Genetics 141(4):1597-1604; Kunkel et al. (1993) Plant Cell 5(8):865-875; Jones et al. (1994) Science 266(5186):789-793; Mindrinos (1994) Cell 78(6):1089-1099; Bent et al. (1994) Science 265(5180):1856-1860; Dixon et al. (1995) Mol. Plant Microbe Interact. 8(2):200-206; Salmeron et al. (1996) Cell 86(1):123-133; Rommens et al. (1995) Plant Cell 7:1537-1544; Buschges et al. (1997) Cell 88(5):695-705; Song et al. (1995) Science 270(5243):1804-1806; Loh et al. (1995) Proc. Natl. Acad. Sci. USA 92(10):4181-4184; Tornero et al. (1996) Plant J. 10(2):315-330; Staskawicz et al. (1995) Science 268(5211):661-667; Whitham et al. (1994) Cell 78(6):1101-1115; Dickinson et al. (1993) Mol. Plant Microbe Interact. 6(3):341-347; Innis et al. (1993) Plant J 4:813-820; Leister et al. (1996) Proc. Natl. Acad. Sci. USA 93(26):15497-15502; Kanazin et al. (1996) Proc. Natl. Acad. Sci. USA 93(21):11746-11750; and Hammond-Kosack et al. (1996) Plant Cell 8(10):1773-1791; the disclosures of which are herein incorporated by reference.

Domain swapping allows for the generation of new resistance specificities. Such newly synthesized resistance genes can be designed for detection of a broad range of pathogen genotypes.

Thus nucleotide sequences of the invention and the proteins or partial-length proteins encoded thereby include the naturally occurring forms as well as variants and fragments thereof. The nucleotide sequences encoding the disease resistance proteins or partial-length proteins of the present invention can be the naturally occurring sequences or they may be synthetically derived sequences. Alternatively, the nucleotide sequences for the disease resistance gene homologues of the present invention can be utilized to isolate homologous disease resistance genes from other plants, including Arabidopsis, sorghum, Brassica, wheat, tobacco, cotton, tomato, barley, sunflower, cucumber, alfalfa, soybeans, sorghum, etc. In this manner, methods such as PCR, hybridization, and the like can be used to identify such sequences based on their sequence homology to the sequences set forth herein. Sequences isolated based on their sequence identity to the entire RGH sequences set forth herein or to fragments thereof are encompassed by the present invention.

In a PCR approach, oligonucleotide primers can be designed for use in PCR reactions to amplify corresponding DNA sequences from cDNA or genomic DNA extracted from any plant of interest. Methods for designing PCR primers and PCR cloning are generally known in the art and are disclosed in Sambrook et al. (1989) Molecular Cloning: A Laboratory Manual (2d ed., Cold Spring Harbor Laboratory Press, Plainview, N.Y.). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York). Known methods of PCR include, but are not limited to, methods using paired primers, nested primers, single specific primers, degenerate primers, gene-specific primers, vector-specific primers, partially-mismatched primers, and the like.

In hybridization techniques, all or part of a known nucleotide sequence is used as a probe that selectively hybridizes to other corresponding nucleotide sequences present in a population of cloned genomic DNA fragments or cDNA fragments (i.e., genomic or cDNA libraries) from a chosen organism. The hybridization probes may be genomic DNA fragments, cDNA fragments, RNA fragments, or other oligonucleotides, and may be labeled with a detectable group such as ³²P, or any other detectable marker. Thus, for example, probes for hybridization can be made by labeling synthetic oligonucleotides based on the RGH sequence of the invention. Methods for preparation of probes for hybridization and for construction of cDNA and genomic libraries are generally known in the art and are disclosed in Sambrook et al. (1989) Molecular Cloning: A Laboratory Manual (2d ed., Cold Spring Harbor Laboratory Press, Plainview, N.Y.).

For example, the entire RGH sequence disclosed herein, or one or more portions thereof, may be used as a probe capable of specifically hybridizing to corresponding disease resistance gene sequences and messenger RNAs. To achieve specific hybridization under a variety of conditions, such probes include sequences that are unique among disease resistance gene sequences and are preferably at least about 10 nucleotides in length, and most preferably at least about 20 nucleotides in length. Such probes may be used to amplify corresponding disease resistance gene sequences from a chosen organism by PCR. This technique may be used to isolate additional coding sequences from a desired organism or as a diagnostic assay to determine the presence of coding sequences in an organism. Hybridization techniques include hybridization screening of plated DNA libraries (either plaques or colonies; see, for example, Sambrook et al. (1989) Molecular Cloning: A Laboratory Manual (2d ed., Cold Spring Harbor Laboratory Press, Plainview, N.Y.).

Hybridization of such sequences may be carried out under stringent conditions. By “stringent conditions” or “stringent hybridization conditions” is intended conditions under which a probe will hybridize to its target sequence to a detectably greater degree than to other sequences (e.g., at least 2-fold over background). Stringent conditions are sequence-dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length, preferably less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C, and a wash in 0.1×SSC at 60 to 65° C.

Specificity is typically the function of post-hybridization washes, the critical factors being the ionic strength and temperature of the final wash solution. For DNA-DNA hybrids, the Tₘ can be approximated from the equation of Meinkoth and Wahl (1984) Anal. Biochem. 138:267-284: Tₘ=81.5° C. +16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1EC for each 1% of mismatching; thus, Tₘ, hybridization, and/or wash conditions can be adjusted to hybridize to sequences of the desired identity. For example, if sequences with ≧90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution), it is preferred to increase the SSC concentration so that a higher temperature can be used. An extensive guide to the hybridization of nucleic acids is found in Tijssen (1993) Laboratory Techniques in Biochemistry and Molecular Biology—Hybridization with Nucleic Acid Probes, Part I, Chapter 2 (Elsevier, N.Y.); and Ausubel et al., eds. (1995) Current Protocols in Molecular Biology, Chapter 2 (Greene Publishing and Wiley-Interscience, New York). See Sambrook et al. (1989) Molecular Cloning: A Laboratory Manual (2d ed., Cold Spring Harbor Laboratory Press, Plainview, N.Y.). In general, sequences that encode a disease resistance protein and hybridize to the RGH sequences disclosed herein will be at least 40% to 50% homologous, about 60% to 70% homologous, and even about 80%, 85%, 90%, 95% to 98% homologous or more with the disclosed RGH sequence. That is, the sequence similarity of sequences may range, sharing at least about 40% to 50%, about 60% to 70%, and even about 80%, 85%, 90%, 95% to 98% sequence similarity.

Generally, since leader peptides are not highly conserved between monocots and dicots, sequences can be utilized from the carboxyterminal end of the protein as probes for the isolation of corresponding sequences from any plant. Nucleotide probes can be constructed and utilized in hybridization experiments as discussed above. In this manner, even gene sequences that are divergent in the aminoterminal region can be identified and isolated for use in the methods of the invention.

Thus the disclosed RGH nucleotide sequences or portions thereof can be used as probes for identifying nucleotide sequences for similar disease resistance genes in a chosen plant or organism. Once similar genes are identified, their respective nucleotide sequences can be utilized in the present invention to enhance disease resistance in a plant.

The following terms are used to describe the sequence relationships between two or more nucleic acids or polynucleotides: (a) “reference sequence”, (b) “comparison window”, (c) “sequence identity”, (d) “percentage of sequence identity”, and (e) “substantial identity”.

(a) As used herein, “reference sequence” is a defined sequence used as a basis for sequence comparison. A reference sequence may be a subset or the entirety of a specified sequence; for example, as a segment of a full-length cDNA or gene sequence, or the complete cDNA or gene sequence.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well known in the art. Optimal alignment of sequences for comparison may be conducted by the local homology algorithm of Smith et al. (1981) Adv. Appl. Math. 2:482; by the homology alignment algorithm of Needleman et al. (1970) J. Mol. Biol. 48:443; by the search for similarity method of Pearson et al. (1988) Proc. Natl. Acad. Sci. 85:2444; by computerized implementations of these algorithms, including, but not limited to: CLUSTAL in the PC/Gene program by Intelligenetics, Mountain View, Calif.; GAP, BESTFIT, BLAST, FASTA, and TFASTA in the Wisconsin Genetics Software Package, Genetics Computer Group (GCG), 575 Science Drive, Madison, Wis., USA; the CLUSTAL program is well described by Higgins et al. (1988) Gene 73:237-244 (1988); Higgins et al. (1989) CABIOS 5:151-153; Corpet et al. (1988) Nucleic Acids Res. 16:10881-90; Huang et al. (1992) Computer Applications in the Biosciences 8:155-65, and Person et al. (1994) Meth. Mol. Biol. 24:307-331; preferred computer alignment methods also include the BLASTP, BLASTN, and BLASTX algorithms (see Altschul et al. (1990) J. Mol. Biol. 215:403-410). Alignment is also often performed by inspection and manual alignment.

(c) As used herein, “sequence identity” or “identity” in the context of two nucleic acid or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

(e)(i) The term “substantial identity” of polynucleotide sequences means that a polynucleotide comprises a sequence that has at least 70% sequence identity, preferably at least 80%, more preferably at least 90%, and most preferably at least 95%, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 60%, more preferably at least 70%, 80%, 90%, and most preferably at least 95%.

Another indication that nucleotide sequences are substantially identical is if two molecules hybridize to each other under stringent conditions. Generally, stringent conditions are selected to be about 5° C. lower than the thermal melting point (Tₘ) for the specific sequence at a defined ionic strength and pH. However, stringent conditions encompass temperatures in the range of about 1° C. to about 20° C., depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.

(e)(ii) The term “substantial identity” in the context of a peptide indicates that a peptide comprises a sequence with at least 70% sequence identity to a reference sequence, preferably 80%, more preferably 85%, most preferably at least 90% or 95% sequence identity to the reference sequence over a specified comparison window. Preferably, optimal alignment is conducted using the homology alignment algorithm of Needleman et al. (1970) J. Mol. Biol. 48:443. An indication that two peptide sequences are substantially identical is that one peptide is immunologically reactive with antibodies raised against the second peptide. Thus, a peptide is substantially identical to a second peptide, for example, where the two peptides differ only by a conservative substitution. Peptides that are “substantially similar” share sequences as noted above except that residue positions that are not identical may differ by conservative amino acid changes.

The RGHs of the present invention, fragments and variants thereof, and any similar sequences identified in other organisms or new resistance gene sequences synthesized by DNA shuffling can be utilized to enhance disease resistance in a plant.

Methods of the invention involve stably transforming a plant with one or more of these novel disease resistance gene homologue nucleotide sequences operably linked with a promoter capable of driving expression of a gene in a plant cell. Expression of the novel disease resistance gene homologues confers disease resistance to a plant by interacting with the complementing phytopathogen avirulence gene product released into the plant by the invading plant pathogen. The plant to be transformed may or may not have preexisting disease resistance genes present in its genome. If so, transformation with one of these novel disease resistance gene homologues further enhances disease resistance of the transformed plant to include resistance to pathogens carrying the complementing avirulence gene.

The plant undergoing transformation with the RGH of the present invention may additionally be transformed with its complementing avr gene operably linked to regulatory regions. The expression of the two genes in the plant cell induces the disease resistance pathway or induces immunity in the plant. That is, the expression of the genes can induce a defense response in the cell or can turn on the disease resistance pathway to obtain cell death. The end result can be controlled by the level of expression of the avr gene in the plant. Where the expression is sufficient to cause cell death, such response is a receptor-mediated programmed response. See, for example, Ryerson and Heath (1996) Plant Cell 8:393-402 and Dangl et al. (1996) Plant Cell 8:1793-1807.

The nucleotide sequences for the disease resistance gene homologues of the present invention are useful in the genetic manipulation of any plant when operably linked to a promoter that is functional within the plant. In this manner, the nucleotide sequences of the invention are provided in expression cassettes for expression in the plant of interest.

Such expression cassettes will include 5′ and 3′ regulatory sequences operably linked to an RGH sequence of the invention. By “operably linked” is intended a functional linkage between a promoter and a second sequence, wherein the promoter sequence initiates and mediates transcription of the DNA sequence corresponding to the second sequence. Generally, operably linked means that the nucleic acid sequences being linked are contiguous and, where necessary to join two protein coding regions, contiguous and in the same reading frame. The cassette may additionally contain at least one additional gene to be cotransformed into the organism. Alternatively, the additional gene(s) can be provided on multiple expression cassettes.

For example, in one embodiment of the invention, the expression cassette may additionally comprise the complementing avr gene for the resistance gene of the present invention operably linked to regulatory regions functional within the plant undergoing transformation. Alternatively, the complementing avr gene may be provided on another expression cassette. Preferably expression of the avr gene would be regulated by an inducible promoter, more preferably a pathogen-inducible promoter. In this manner, invasion of the plant by a nonspecific pathogen triggers expression of the avr gene. The avr gene product would then interact with the product of the introduced complementing resistance gene, whose expression may be under the control of a constitutive or inducible promoter. This specific recognition event would activate a cascade of plant resistance-related genes, leading to a hypersensitive response in the invaded cells and inhibition of further spread of the pathogen beyond the site of initial infection. Extent of the disease resistance response could be manipulated by altering expression of the avr gene via its promoter sequence, as disclosed in the co-pending application entitled “Methods for Enhancing Disease Resistance in Plants,” U.S. Patent Application Serial No. 60/076,151, filed Feb. 26, 1998, herein incorporated by reference.

Such an expression cassette is provided with a plurality of restriction sites for insertion of the RGH sequence to be under the transcriptional regulation of the regulatory regions. The expression cassette may additionally contain selectable marker genes.

The expression cassette will include in the 5′-3′ direction of transcription, a transcriptional and translational initiation region, an RGH sequence of the invention, and a transcriptional and translational termination region functional in plants. The transcriptional initiation region, the promoter, may be native or analogous or foreign or heterologous to the plant host. Additionally, the promoter may be the natural sequence or alternatively a synthetic sequence. By “foreign” is intended that the transcriptional initiation region is not found in the native plant into which the transcriptional initiation region is introduced or alternatively is found after transformation at a different site in the genome. As used herein, a chimeric gene comprises a coding sequence operably linked to a transcription initiation region that is heterologous to the coding sequence.

While it may be preferable to express the sequences using heterologous promoters, the native promoter sequences may be used. Such constructs would change expression levels of the RGH protein in the plant or plant cell. Thus, the phenotype of the plant or plant cell is altered.

A number of promoters can be used in the practice of the invention, including constitutive, pathogen-inducible, wound-inducible, and tissue-specific promoters. In the event that continuous resistance to a pathogen carrying the complementing avr gene is desirable, a constitutive promoter is preferable. Such constitutive promoters include, for example, the core promoter of the Rsyn7 (copending U.S. application Ser. No. 08/661,601); the core CaMV 35S promoter (Odell et al. (1985) Nature 313:810-812); rice actin (McElroy et al (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. application Ser. No. 08/409,297), and the like. Other constitutive promoters include, for example, U.S. Pat. Nos. 5,608,149; 5,608,144; 5,604,121; 5,569,597; 5,466,785; 5,399,680; 5,268,463; and 5,608,142; and the co-pending application entitled “Constitutive Maize Promoters,” U.S. patent application Ser. No. 09/257,584, filed Feb. 25, 1999, herein incorporated by reference.

Alternatively, it may be desirable to have the introduced RGH sequence expressed upon pathogen invasion, wherein expression of the resistance gene results in the plant being primed in the event of invasion by the pathogen carrying the complementing avr gene. Such pathogen-inducible promoters include those from pathogenesis-related proteins (PR proteins), which are induced following infection by a pathogen; e.g., PR proteins, SAR proteins, beta-1,3-glucanase, chitinase, etc. See, for example, Redolfi et al (1983) Neth. J. Plant Pathol. 89:245-254; Uknes et al. (1992) The Plant Cell 4:645-656; and Van Loon (1985) Plant Mol. Virol. 4:111-116. See also the co-pending application entitled “Inducible Maize Promoters”, U.S. patent application Ser. No. 09/257,583, filed Feb. 25, 1999, and herein incorporated by reference.

Of interest are promoters that are expressed locally at or near the site of pathogen infection. See, for example, Marineau et al. (1987) Plant Mol. Biol. 9:335-342; Matton et al. (1989) Molecular Plant-Microbe Interactions 2:325-331; Somsisch et al. (1986) Proc. Natl. Acad. Sci. USA 83:2427-2430; Sommsich et al. (1988) Mol. Gen. Genet.2:93-98; and Yang, Y (1996) Proc. Natl. Acad. Sci. USA 93:14972-14977. See also, Chen et al. (1996) Plant J. 10:955-966; Zhang and Sing (1994) Proc. Natl. Acad. Sci. USA 91:2507-2511; Warner et al. (1993) Plant J. 3:191-201; Siebertz et al. (1989) Plant Cell 1:961-968; and the references cited therein. Of particular interest is the inducible promoter for the maize PRms gene, whose expression is induced by the pathogen Fusarium moniliforme (see, for example, Cordero et al. (1992) Physiol.

Additionally, as pathogens find entry into plants through wounds or insect damage, a wound-inducible promoter may be used in the constructions of the invention. Such wound-inducible promoters include potato proteinase inhibitor (pin II) gene (Ryan (1990) Ann. Rev. Phytopath. 28:425-449; Duan et al. (1996) Nature Biotechnology 14:494-498); wun1 and wun2, U.S. Pat. No. 5,428,148; win1 and win2 (Stanford et al. (1989) Mol. Gen. Genet. 215:200-208); systemin (McGurl et al. (1992) Science 225:1570-1573); WIP1 (Rohmeier et al. (1993) Plant Mol. Biol. 22:783-792; Eckelkamp et al. (1993) FEBS Letters 323:73-76); MPI gene (Corderok et al. (1994) Plant J. 6(2):141-150); and the like, herein incorporated by reference.

Where expression of the introduced gene is preferred within a particular tissue that is susceptible to attack by the pathogen carrying the complementing avr gene, a tissue-specific promoter may be desirable. Tissue specific promoters include Yamamoto et al. (1997) Plant J. 12(2)255-265; Kawamata et al. (1997) Plant Cell Physiol. 38(7):792-803; Hansen et al. (1997) Mol Gen Genet. 254(3):337-343; Russell et al. (1997) Transgenic Res. 6(2):157-168; Rinehart et al. (1996) Plant Physiol. 112(3):1331-1341; Van Camp et al. (1996) Plant Physiol. 112(2):525-535; Canevascini et al. (1996) Plant Physiol. 112(2):513-524; Yamamoto et al. (1994) Plant Cell Physiol. 35(5):773-778; Lam (1994) Results Probl Cell Differ. 20:181-196; Orozco et al. (1993) Plant Mol Biol. 23(6):1129-1138; Matsuoka et al. (1993) Proc Natl Acad Sci U S A 90(20):9586-9590; and Guevara-Garcia et al. (1993) Plant J. 4(3):495-505.

Any of these promoters can be modified, if necessary, for weak expression. Such weak promoters cause background levels of the disease resistance protein to be expressed. Generally, by “weak promoter” is intended either a promoter that drives expression of a coding sequence at a low level. By low level is intended at levels of about 1/1000 transcripts to about 1/100,000 transcripts to about 1/500,000 transcripts. Alternatively, it is recognized that weak promoters also encompasses promoters that are expressed in only a few cells and not in others to give a total low level of expression. Where a promoter is expressed at unacceptably high levels, portions of the promoter sequence can be deleted or modified to decrease expression levels. Such weak constitutive promoters include, for example, the core promoter of the Rsyn7 (copending application Ser. No. 08/661,601), the core 35S CaMV promoter, and the like.

Thus the expression cassette will include in the 5′-3′ direction of transcription, a transcriptional and translational initiation region, a nucleotide sequence encoding the particular disease resistance protein of the present invention, and a transcriptional and translational termination region functional in plants. The termination region may be native with the transcriptional initiation region, may be native with the DNA sequence of interest, or may be derived from another source. Convenient termination regions are available from the Ti-plasmid of A. tumefaciens, such as the octopine synthase and nopaline synthase termination regions. See also, Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. 1989) Nucleic Acids Res. 17:7891-7903; Joshi et al. (1987) Nucleic Acid Res. 15:9627-9639.

Where appropriate, the RGH sequence and any additional gene(s) may be optimized for increased expression in the transformed plant. That is, these nucleotide sequences can be synthesized using plant-preferred codons for improved expression. Methods are available in the art for synthesizing plant-preferred genes. See, for example, U.S. Pat. Nos. 5,380,831, 5,436, 391, and Murray et al. (1989) Nucleic Acids Res. 17:477-498, herein incorporated by reference.

Additional sequence modifications are known to enhance gene expression in a cellular host. These include elimination of sequences encoding spurious polyadenylation signals, exon-intron splice site signals, transposon-like repeats, and other such well-characterized sequences which may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

The expression cassettes may additionally contain 5′ leader sequences in the expression cassette construct. Such leader sequences can act to enhance translation. Translation leaders are known in the art and include: picomavirus leaders, for example, EMCV leader (Encephalomyocarditis 5′ noncoding region) (Elroy-Stein et al. (1989) PNAS USA 86:6126-6130); potyvirus leaders, for example, TEV leader (Tobacco Etch Virus) (Allison et al. (1986); MDMV leader (Maize Dwarf Mosaic Virus); Virology 154:9-20), and human immunoglobulin heavy-chain binding protein (BiP), (Macejak et al. (1991) Nature 353:90-94); untranslated leader from the coat protein mRNA of alfalfa mosaic virus (AMV RNA 4) (Jobling et al. (1987) Nature 325:622-625); tobacco mosaic virus leader (TMV) (Gallie et al. (1989) in Molecular Biology of RNA, ed. Cech (Liss, N.Y.), pp. 237-256); and maize chlorotic mottle virus leader (MCMV) (Lommel et al. (1991) Virology 81:382-385). See also, Della-Cioppa et al. (1987) Plant Physiol. 84:965-968. Other methods known to enhance translation can also be utilized, for example, introns, and the like.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g. transitions and transversions may be involved.

The RGH sequences of the present invention can be used to transform any plant. In this manner, genetically modified plants, plant cells, plant tissue, seed, and the like can be obtained. Transformation protocols may vary depending on the type of plant or plant cell, i.e., monocot or dicot, targeted for transformation. Suitable methods of introducing nucleotide sequences into plant cells and subsequent insertion into the plant genome include microinjection (Crossway et al. (1986) Biotechniques 4:320-334), electroporation (Riggs et al. (1986) Proc. Natl. Acad. Sci. USA 83:5602-5606, Agrobacterium-mediated transformation (Townsend et al., U.S. Pat. No. 5,563,055), direct gene transfer (Paszkowski et al. (1984) EMBO J. 3:2717-2722), and ballistic particle acceleration (see, for example, Sanford et al., U.S. Pat. No. 4,945,050; Tomes et al. (1995) “Direct DNA Transfer into Intact Plant Cells via Microprojectile Bombardment,” in Plant Cell, Tissue, and Organ Culture: Fundamental Methods, ed. Gamborg and Phillips (Springer-Verlag, Berlin); and McCabe et al. (1988) Biotechnology 6:923-926). Also see Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P:175-182 (soybean); Singh et al. (1998) Theor. Appl. Genet. 96:319-324 (soybean); Datta et al. (1990) Biotechnology 8:736-740 (rice); Klein et al. (1988) Proc. Nat. Acad. Sci. USA 85:4305-4309 (maize); Klein et al. (1988) Biotechnology 6:559-563 (maize); Tomes, U.S. Pat. No. 5,240,855; Buising et al., U.S. Pat. Nos. 5,322,783 and 5,324,646; Tomes et al. (1995) “Direct DNA Transfer into Intact Plant Cells via Microprojectile Bombardment,” in Plant Cell, Tissue, and Organ Culture: Fundamental Methods, ed. Gamborg (Springer-Verlag, Berlin) (maize); Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; Bowen et al., U.S. Pat. No. 5,736,369 (cereals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, N.Y.), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); all of which are herein incorporated by reference.

The cells that have been transformed may be grown into plants in accordance with conventional ways. See, for example, McCormick et al. (1986) Plant Cell Reports 5:81-84. These plants may then be grown, and either pollinated with the same transformed strain or different strains, and the resulting hybrid having the desired phenotypic characteristic identified. Two or more generations may be grown to ensure that the subject phenotypic characteristic is stably maintained and inherited and then seeds harvested to ensure the desired phenotype or other property has been achieved.

The methods of the invention can be used with other methods available in the art for enhancing disease resistance in plants.

The following examples are offered by way of illustration and not by way of limitation.

### Experimental

In the past few years, many R genes have been cloned from various plant species. Sequence analysis has shown that some conserved structural features are common among cloned R genes which confer resistance to bacterial, fungal, viral, and nematode pathogens (Staskawicz et al. (1995) Science 268:661-667; Bent (1996) Plant Cell 8:1757-1771; Baker et al. (1997) Science 276:726-733). As predicted by the gene-for-gene theory, the R gene products contain various important domains for interacting with pathogen elicitors (see review by Bent (1996) Plant Cell 8:1757-1771). Leucine-rich repeats (LRRs), which are the least conserved domains among R genes, are believed to be responsible for protein-protein interaction and thus might be involved in pathogen recognition and specificity. Recently, studies of two groups strongly support such a role for LRRs (Anderson et al. (1997) Plant Cell 9:641-651; Ori et al. (1997) Plant Cell 9:521-532). The presence of three conserved subdomains in nucleotide-binding site (NBS) domains, i.e., P-loop, kinase-2, and kinase-3a, suggests that ATG/GTP binding is essential for the function of some R gene products. LRRs and NBS are also thought to be involved in signaling at some level in the disease resistance signaling pathway (Baker et al. (1997) Science 276:726-733). A domain having some similarity to the cytoplasmic signaling domain of Toll/interleukin-1 (TIR) receptors is also present in some R gene products. Leucine zippers (LZ), which have a role in homo- and heterodimerization of eukaryotic transcription factors, are also found some R gene products. Serine/threonine kinase is part of some R gene products, suggesting an involvement of these proteins in the activation of resistance-related genes in signaling pathways.

To date, cloned R genes could be classified into several groups based on these conserved structural domains in their gene products. In the NBS-LRR superfamily, tobacco N, flax L6 and M, and Arabidopsis RPP5 have TIR on the amino-terminus of their gene products (Whitham et al. (1994) Cell 78:1101-1115; Lawrence et al. (1995) Plant Cell 7:1195-1206; Anderson et al. (1997) Plant Cell 19:641-651; Parker et al. (1997) Plant Cell 9:879-894), and Arabidopsis RPM1 and RPS2 and tomato Prf have LZ on the amino-terminus (Bent et al. (1994) Plant Cell 8:1757-1771; Mindrinos et al. (1994) Cell 78:1089-1099; Grant et al. (1995) Science 269:843-846; Salmeron et al. (1996) Cell 86:123-133). Tomato Cf-9 and Cf-2 and sugar beet HS1^{pro-1} belong to a group of R genes that have LRRs and a transmembrane domain (Jones et al. (1994) Science 266:789-793; Dixon et al. (1996) Cell 84:451-459; Cai et al. (1997) Science 275:832-834). Tomato Pto is a kinase-encoding R gene (Martin et al. (1993) Science 262:1432-1436), and wheat Lr10 has a transmembrane domain in addition to its kinase domain (Feuillet et al. (1997) Plant J. 11:45-52). Rice Xa21 is the only R gene encoding a LRR receptor kinase with a transmembrane region between LRRs and the kinase domain (Song et al. (1995) Science 270:1804-1806).

Recently, some groups have successfully isolated R gene candidates using PCR with primers designed based on highly conserved motifs among cloned R genes (Kanazin et al. (1996) Proc. Natl. Acad. Sci. USA 93:11746-11750; Leister et al. (1996) Nature Gen. 14:421-429, Yu et al. (1996) Proc. Natl. Acad. Sci. USA 93:11751-11756). Most of the PCR clones isolated using this approach contain some other conserved motifs besides those contributed by the primers. Some of the PCR clones have also been mapped near to and are potentially linked to known R genes. These results suggest that sufficient sequence differences exist between R genes and other sequences, and therefore, the PCR approach is useful for isolating R gene candidates. In addition, a gene encoding a new receptor-like kinase has been successfully cloned in wheat using a homologue probe of serine/threonine kinase genes (Feuillet et al. (1997) Plant J. 11:45-52). Therefore, isolating R gene candidates based on conserved motifs of cloned R genes is a practical approach among wide plant taxa.

In Example 1, a PCR approach was used to isolate resistance gene homologues (RGHs) from maize, sorghum, and rice. Thirteen RGH families were isolated and genetically mapped to the corresponding plant genomes. The corresponding gene of one RGH has also been isolated and shown to be a member of the LZ-NBS-LRR family. Example 2 demonstrates use of the RGH sequences for transformation of a plant to enhance disease resistance.

### EXAMPLE 1

### PCR Amplification, Cloning, and Sequence Analysis of RGHs

Degenerate primers LM638 and LM637, which were designed from the conserved P-loop and the putative transmembrane sequences (Kanazin et al. (1996) Proc. Natl. Acad. Sci. USA 93:11746-11750), respectively, were used for amplification of RGHs by PCR. Source DNA for RGH amplification included maize genomic DNA (Q66), maize total cDNA prepared from root (Lhad2), leaf (Lhad2), and two-leaf seedling (corn B73 cDNA library, Clontech Laboratories, Inc.); sorghum genomic DNA (BT×623); and rice genomic DNA (TQ). A 100-μl PCR cocktail containing 80 ng of source DNA, 20 pmol each of the primers, 5 units of Taq polymerase (Promega), 1×Taq polymerase reaction buffer (Promega), 2.5 mM MgCl₂, and 0.2 mM each of dATP, dTTP, dGTP, and dCTP was subjected to 35 cycles of PCR amplification as described in Kanazin et al. (1996) Proc. Natl. Acad. Sci. USA 93:11746-11750). To enrich the variability of RGH PCR products, the concentration of MgCl₂ was varied from to 2.5 to 6.0 mM with or without the addition of DMSO to a final concentration of 5%. Amplification products were cloned into pBluescript KS (Stratagene) or pGEM-T Easy (Promega) vector and sequenced using the Pharmacia Biotech model ALF Express automated sequencer. DNA sequences were analyzed using GCG (University of Wisconsin Genetics Computer Group, Madison) sequence analysis packages. Alignments of amino acid sequences were carried out using the PILEUP function, and phylogenetic analysis was done using the DISTANCES and GROWTREE functions.

BAC Library Screening and Sequence Analysis of an RGH

Two maize RGH clones, M5-1 and M6-1, were used to screen a sorghum BAC library (BT×623) and two rice BAC libraries (Teqing and Lemont) by the techniques described before (Woo et al. (1994) Nucleic Acids Res. 22:4922-4931). One RGH was subcloned from a sorghum BAC clone selected with M6-1 into a pBluescript KS vector with a Bam HI+Sal I double digestion and sequenced using the transposon-facilitated sequencing strategy (Strathmann et al. (1991) Proc. Natl. Acad. Sci. USA 88:1247-1250).

Cloning and Sequence Analysis of RGHs Isolated from Maize, Sorghum, and Rice

The results of PCR amplification, cloning, and classification are summarized in Table 1. By using primers LM638 and LM637 under varied conditions, several PCR products showing sequence homology to cloned R genes were identified in maize, sorghum, and rice. PCR products of 0.5, 0.6, and 1.1 kb were amplified from maize genomic DNA. The 0.5 and 0.6-kb maize PCR products were cloned and analyzed.

TABLE 1
PCR Amplification, Cloning, and Families
of Maize, Sorghum, and Rice RGHs.

| Source DNA | PCR products | RGH families^{a} |
|---|---|---|
| Maize | | |
| Genomic DNA | 0.5 kb | M5-1 |
| | 0.6 kb | M6-1 |
| | 1.1 kb | N.A.^{b} |
| cDNA from: | | |
| root | 0.5 kb | M5-1, M5-6 |
| seedling | 0.7 kb | —^{c} |
| leaf | 0.5 kb | M5-1, M5-6 |
| | 0.7 kb | N.A.^{b} |
| Sorghum | | |
| genomic DNA | 0.5 kb | S5-2A, S5-2B, S5-5 |
| | 0.6 kb | S6-1, S6-2 |
| | 0.8 kb | —^{c} |
| | 1.1 kb | S11-1 |
| Rice | | |
| genomic DNA | 0.5 kb | R5-1, R5-2, R5-3, R5-4 |

^{a}RGH clones were classified based on results of cross-hybridization under high stringency condition (0.1XXXC, 0.1% SDS) and sequencing;
^{b}N.A., not analyzed;
—^{c}, no RGH identified.

Two families of maize RGHs, M5-1 (SEQ ID NO: 1) and M6-1 (SEQ ID NO: 3), were identified in these PCR products based on the results of cross-hybridization under high stringency conditions (0.1×SSC, 0.2% SDS) and sequencing. The predicted partial-length proteins encoded by these maize RGHs are set forth in SEQ ID NOS: 2 and 5, respectively. PCR products of 0.5 and/or 0.7 kb were amplified from maize cDNA prepared from root, seedling, and leaf, and M5-6 (SEQ ID NO: 6) was identified in the 0.5-kb PCR products in addition to M5-1. The predicted partial-length protein encoded by M5-6 is set forth in SEQ ID NO: 7.

A heterogeneous 0.5-kb PCR product was amplified from rice genomic DNA under regular PCR condition without DMSO and four rice RGH families (R5-1, SEQ ID NO: 8; R5-2, SEQ ID NO: 10; R5-3, SEQ ID NO: 12; and R5-4, SEQ ID NO: 14) were identified. The predicted partial-length proteins encoded by these rice RGHs are set forth in SEQ ID NOS: 9, 11, 13, and 15, respectively.

PCR products of 0.5, 0.6, 0.8, and 1.1 kb were amplified from sorghum genomic DNA. Six sorghum RGH families (S5-5, SEQ ID NO: 16; S5-2A, SEQ ID NO: 18; S5-2B, SEQ ID NO: 20; S6-1, SEQ ID NO: 21; S6-2, SEQ ID NO: 24; and S11, having four members referred to as S11-1 (SEQ ID NO: 27), S11-25 (SEQ ID NO: 30), S11-27 (SEQ ID NO: 31), and S11-34 (SEQ ID NO: 32) were identified in these PCR products except the 0.8-kb product. The predicted partial-length proteins encoded by S5-5, S5-2A, S6-1, S6-2, and S11-1 are set forth in SEQ ID NOS: 17, 19, 23, 26, and 29, respectively.

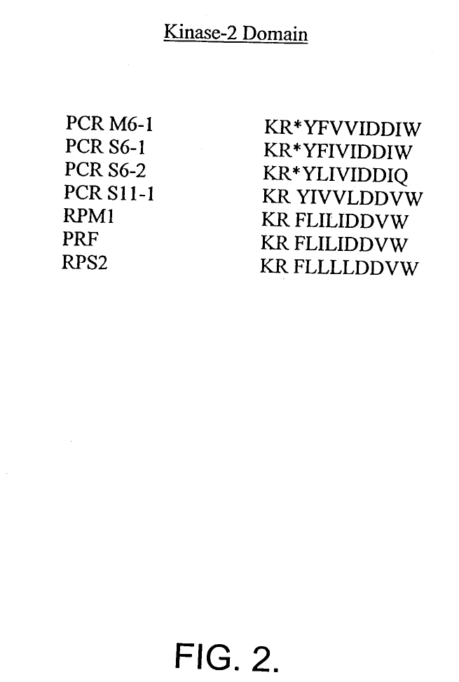

At least one clone from each RGH family was sequenced. The deduced amino acid sequences were highly conserved and showed striking homology to cloned R genes, particularly to Arabidopsis RPM1 and RPS2 and tomato Prf (FIGS. 1A-1B). Despite the size difference, all the RGH families identified had highly conserved kinase-2 and kinase-3a domains shared by R genes in the NBS-LRR superfamily in addition to P-loop and the putative transmembrane domain that were contributed by the primers. However, RGH families S6-1, S6-2 and S11-1 cannot be translated into polypeptides uninterrupted by stop codons and frameshifts were found in the sequences. When the sequences near the frameshifts were checked carefully, a putative intron was found within the kinase-2 domain coding sequences (data not shown; FIG. 2). Although RGH M6-1 could be translated into a polypeptide without interruption by stop codons, a putative intron was also found within the kinase-2 domain coding region. The size of the putative intron is 126 bp in M6-1 (nucleotides (nt) 211-336 of SEQ ID NO: 3), 92 bp in S6-1 (nt 220-311 of SEQ ID NO: 21), 100 bp in S6-2 (nt 229-328 of SEQ ID NO: 24), and 518 bp in S11-1 (nt 225-742 of SEQ ID NO: 27) (data not shown). Splicing of the putative introns results in coding sequences (SEQ ID NOS: 4, 22, 25, and 28, respectively) that are translated into the predicted partial-length proteins set forth in SEQ ID NOS: 5, 23, 26, and 29, respectively. This splicing gives these RGHs a similar size to cloned R genes and their homologues on the region between P-loop and the putative transmembrane domain (data not shown), as well as aligns the deduced amino acid sequences on kinase-2 domains of these RGHs and cloned R genes perfectly (FIG. 2).

Due to the usage of the degenerate primers in PCR amplification of RGHs and to the presence of a putative intron in some RGH clones, an alignment of amino acid sequences of all RGHs was done starting from the third amino acid of kinase-2 domain (FIGS. 1A-1B). The corresponding regions of Arabidopsis RMP1 and RPS2 and tomato Prf, tobacco N, and flax L6 were also included on the alignment for comparison. Based on this alignment, neighbor-joining method in DISTANCES function was used for analysis of sequence differences and a phylogenetic tree was constructed using the GROWTREE function (data not shown).

Genetic Mapping

M6-1 was mapped to maize chromosome bin 3.04, a region where several known R genes cluster, and is very close to Wsm2.

BAC Screening and Sequence Analysis of RGH S6-1

RGHs M5-1 (SEQ ID NO: 1) and M6-1 (SEQ ID NO: 3) were used as probes to screen sorghum (BT×623) BAC library and two rice (Lemont and Teqing) BAC libraries. Two rice Lemont BACs and three sorghum BACs were identified with M5-1 and M6-1, respectively (data not shown). Copy number of each RGH sequence was determined by digesting the BACs with Hae III, which does not have recognition sites within M5-1 and M6-1 sequences and then by hybridizing with individual RGH probes. One or two copies of M5-1 and one copy of M6-1 were contained in the BACs (data not shown).

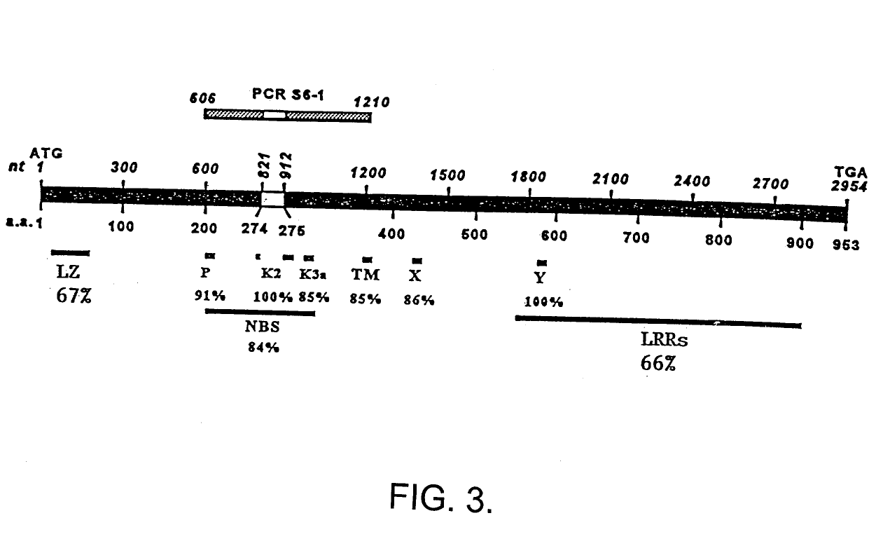

Since M6-1 was mapped near to and is probably linked to Wsm2, subcloning and sequencing of its corresponding gene in one of the BACs were further carried out. A 10-kb BAM HI−Sal I sorghum BAC fragment containing M6-1 sequence was subcloned and sequenced approximately 7.2 kb from the Bam HI end. Sequence analysis indicated that, if not including the P-loop and the putative transmembrane domain contributed by the degenerate PCR primers, the corresponding region for PCR amplification of all RGHs in this sorghum BAC had a DNA sequence similarity of 99.7% and an amino acid sequence similarity of 100% to the PCR RGH family S6-1 (SEQ ID NO: 21) (data not shown). The same 92-bp putative intron found in the kinase-2 domain coding sequence of the PCR RGH family S6-1 was also present in the corresponding region in the BAC subclone (FIG. 3). This putative gene is apparently the corresponding gene or a very close homologue of S6-1 and hereafter referred to as the S6-1 gene. The S6-1 gene (residing within nt 3376-6329 of the BAC clone sequence set forth in SEQ ID NO: 33) is set forth in SEQ ID NO: 34. Removal of the putative 92-bp intron (nt 822-913 of SEQ ID NO: 34) results in a resistance gene having an open-reading frame of 2859 bp (see SEQ ID NO: 35). This 2859-bp region could be translated into a polypeptide of 953 amino acids (SEQ ID NO: 36) without interruption by stop codons (FIG. 3). No apparent continuous coding region could be found in the upstream 3.3-kb region of the putative initiation codon (nt 3376-3378 of SEQ ID NO: 33) or in the downstream 0.8-kb region of the putative stop codon (nt 6327-6329 of SEQ ID NO: 33) (data not shown). The deduced amino acid sequence of the 2859-bp coding region had each of the highly conserved domains present in Arabidopsis RPM1 and RPS2 and tomato Prf, i.e., LZ, P-loop, kinase-2, kinase-3a, a putative transmembrane domain, and LRRs (FIG. 3). In addition, an amino acid sequence alignment of S6-1, Arabidopsis RPM1 and RPS2, and tomato Prf further revealed two other conserved domains of unknown function X and Y. The deduced amino acid sequence (SEQ ID NO: 36) encoded by the S6-1 gene is very similar to that of Arabidopsis RPM1 with a similarity of 67% on LZ, 84% on the overall NBS region, 91% on P-loop, 100% on kinase-2, 85% on kinase-3a, 85% on the putative transmembrane domain, 86% on domain X, 100% on domain Y, and 66% on LRRs (FIG. 3).

Discussion

By using a PCR approach, 13 RGH families have been isolated from maize, sorghum, and rice. Although six to eleven RGH families were isolated from dicots under standard PCR conditions (Kanazin et al. (1996) Proc. Natl. Acad. Sci. USA 93:11746-11750; Leister et al. (1996) Nature Genetics 14:421-429; Yu et al. (1996) Proc. Natl. Acad. Sci. USA 93:11751 -11756), not as many RGH families were identified in maize, sorghum, and rice using a same PCR approach even though various PCR conditions have been tried. Only three RGH families were isolated from different maize sources of DNA under various PCR conditions (M5-1, M6-1, and M5-6), six families from sorghum genomic DNA under various PCR conditions (S5-5, S5-2A, S5-2B, S6-1, S6-2, and S11 (of which S11-1, S 11-25, S 11-27, and S 11-34 are members), and four families from rice genomic DNA under regular PCR conditions (R5-1, R5-2, R5-3, and R5-4) (Table 1). These results suggest that there might not be as many RGH families that belong to the NBS-LRR superfamily in these crops as in soybean and potato (Kanazin et al. (1996) Proc. Natl. Acad. Sci. USA 93:11746-11750; Leister et al. (1996) Nature Genetics 14:421-429; Yu et al. (1996) Proc. Natl. Acad. Sci. USA 93:11746-11750). Alternatively, and most likely, sequences coding for P-loop and the putative transmembrane domains of most RGHs might have diverged or even been lost in these monocots. Since the two degenerate primers used in the PCR amplification were designed from the conserved motifs in R genes cloned from dicots, they might not be able to amplify many of the RGHs in monocots. A phylogenetic tree based on an alignment of the deduced amino acid sequences of several cloned R genes and RGHs isolated from maize, sorghum, and rice also revealed a closer relationship of most RGHs of the same size to each other than to the R genes cloned from dicots (data not shown).

Interestingly, unlike all the cloned R genes in the NBS-LRR superfamily and the majority of their homologues isolated from soybean, potato, and rice (Table 1) having an approximately 0.5-kb continuous coding sequence in the region between the P-loop and the putative transmembrane domain, four RGH families of 0.6 kb or 1.1 kb (M6-1, S6-1, S6-2, S11-1) could not be translated into polypeptides without interruption by stop codons. A putative intron of 92-518 bp was found within the kinase-2 domain coding sequences of these RGHs. Removal of these putative introns would make the deduced amino acid sequences of these RGHs align perfectly with the R genes and RGHs in the same superfamily on kinase-2 domain (FIG. 2), as well as give a similar size on the corresponding regions between the P-loop and the putative transmembrane domain (data not shown). Analysis of the corresponding cDNA of these RGHs will reveal whether or not these introns are real. Some cloned R genes have been shown to have introns; however, no intron within a kinase-2 domain has been reported before.

Of the 13 RGH families isolated, three families were mapped near to known R genes. M6-1 and S6-1 were mapped to maize chromosomal bin 3.04. Several known R genes and quantitative trait loci (QTL) are mapped on this region: Rp3 (common rust), Mv1 (maize mosaic virus), Wsm2 (wheat streak mosaic virus), a QTL associated with resistance to European corn borer, and a QTL associated with resistance to fusarium stalk rot (McMullen and Simcox (1995) Microbe Interactions 6:811-815). Preliminary mapping results suggest that M6-1 is closer to Wsm2 than to Rp3, but how well M6-1 co-segregates with Wsm2 is not known at this point. M5-6 was mapped to maize chromosomal bin 7.04 where another European corn borer QTL is located. However, further information about whether or not M5-6 co-segregates with this QTL is still needed. Most of the RGHs isolated were not mapped to locations where known R genes or QTLs mapped. This is largely due to the lack of phenotypic data of disease reaction. Particularly, there is not yet a good mapping system of sorghum R genes, and not many R genes and useful markers have been mapped on the sorghum genome.

To confirm the identity of RGH M6-1 as an R gene candidate and to learn more about its sequence features, subcloning and sequencing of the corresponding gene in one of the BACs were further carried out. Sequencing results indicated that the corresponding gene of M6-1 in the sorghum BAC was indeed the S6-1 gene. The same putative 92-bp intron found in the PCR RGH family S6-1 (SEQ ID NO: 21) was also present on the same region in the S6-1 gene (FIG. 3). The sequence of S6-1 (SEQ ID NO: 34) obtained from the sorghum BAC subclone (SEQ ID NO: 33) showed that this putative 2954-bp R gene candidate could encode a polypeptide of 953 amino acids (SEQ ID NO: 36) with interruption by the putative 92-bp intron, and its deduced amino acid sequence had all the conserved domains shared by members of the LZ-NBS-LRR R gene family (FIG. 3). Similarity of S6-1 to Arabidopsis RPM1 on the conserved domains was particularly high (66% to 100%) (FIG. 3). The size of this putative gene and the position of all the conserved domains also matched that of Arabidopsis RPM1 and RPS2 perfectly. However, whether the region obtained and sequenced is a full S6-1 gene or whether there are other coding regions in its upstream and/or downstream positions is not known at this point due to the lack of analysis on the corresponding cDNA.

This study, along with the results obtained by other groups (Kanazin et al. 1996; Leister et al. 1996; Yu et al. 1996; Feuillet et al. 1997), indicates that homology-based isolation of R gene candidates could be beneficial to cloning or positioning real R genes conferring resistance to bacterial, fungal, viral, and nematode pathogens. However, whether or not these RGHs are functional R genes conferring resistance to any known or unknown pathogens of maize, sorghum, or rice is not known. Even for RGHs mapped near to known R genes, cDNA analysis and detailed co-segregation tests are still needed, and further DNA complementation transformation testing is also necessary to confirm their role in disease resistance.

### EXAMPLE 2

### Transformation and Regeneration of Transgenic Plants

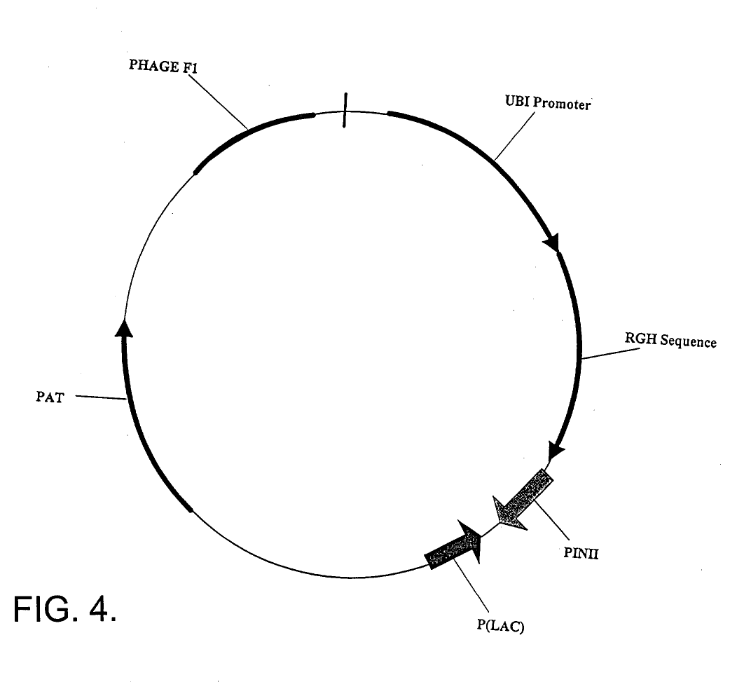

Immature maize embryos from greenhouse donor plants are bombarded with a plasmid containing one of the RGH sequences of the invention operably linked to the ubiquitin (UBI) promoter (FIG. 4) plus a plasmid containing the selectable marker gene PAT (Wohlleben et al. (1988) Gene 70:25-37) that confers resistance to the herbicide Bialaphos. Transformation is performed as follows. All media recipes are in the Appendix.

Preparation of Target Tissue

The ears are surface sterilized in 30% Chlorox bleach plus 0.5% Micro detergent for 20 minutes, and rinsed two times with sterile water. The immature embryos are excised and placed embryo axis side down (scutellum side up), 25 embryos per plate, on 560Y medium for 4 hours and then aligned within the 2.5-cm target zone in preparation for bombardment.

Preparation of DNA

A plasmid vector comprising one of the RGH seuqences of the invention operably linked to the ubiquitin promoter is made. This plasmid DNA plus plasmid DNA containing a PAT selectable marker is precipitated onto 1.1 μm (average diameter) tungsten pellets using a CaCl₂ precipitation procedure as follows:

100 μl prepared tungsten particles in water

10 μl (1 μg) DNA in TrisEDTA buffer (1 μg total)

100 μl 2.5MCaCl₂

10 μl 0.1 M spermidine

Each reagent is added sequentially to the tungsten particle suspension, while maintained on the multitube vortexer. The final mixture is sonicated briefly and allowed to incubate under constant vortexing for 10 minutes. After the precipitation period, the tubes are centrifuged briefly, liquid removed, washed with 500 ml 100% ethanol, and centrifuged for 30 seconds. Again the liquid is removed, and 105 μl 100% ethanol is added to the final tungsten particle pellet. For particle gun bombardment, the tungsten/DNA particles are briefly sonicated and 10 μl spotted onto the center of each macrocarrier and allowed to dry about 2 minutes before bombardment.

Particle Gun Treatment

The sample plates are bombarded at level #4 in particle gun #HE34-1 or #HE34-2. All samples receive a single shot at 650 PSI, with a total of ten aliquots taken from each tube of prepared particles/DNA.

Subsequent Treatment

Following bombardment, the embryos are kept on 560Y medium for 2 days, then transferred to 560R selection medium containing 3 mg/liter Bialaphos, and subcultured every 2 weeks. After approximately 10 weeks of selection, selection-resistant callus clones are transferred to 288J medium to initiate plant regeneration. Following somatic embryo maturation (2-4 weeks), well-developed somatic embryos are transferred to medium for germination and transferred to the lighted culture room. Approximately 7-10 days later, developing plantlets are transferred to 272V hormone-free medium in tubes for 7-10 days until plantlets are well established. Plants are then transferred to inserts in flats (equivalent to 2.5″ pot) containing potting soil and grown for 1 week in a growth chamber, subsequently grown an additional 1-2 weeks in the greenhouse, then transferred to classic 600 pots (1.6 gallon) and grown to maturity. Plants are monitored and scored for disease resistance.

APPENDIX

| | | |
|---|---|---|
| INGREDIENT | AMOUNT | UNIT |

272 V

| | | |
|---|---|---|
| D-I H₂O | 950.000 | Ml |
| MS Salts (GIBCO 11117-074) | 4.300 | G |
| Myo-Inositol | 0.100 | G |
| MS Vitamins Stock Solution ## | 5.000 | Ml |
| Sucrose | 40.000 | G |
| Bacto-Agar @ | 6.000 | G |

Directions:
@ = Add after bringing up to volume
Dissolve ingredients in polished D-I H₂O in sequence
Adjust to pH 5.6
Bring up to volume with polished D-I H₂O after adjusting pH
Sterilize and cool to 60° C.
## = Dissolve 0.100 g of Nicotinic Acid; 0.020 g of Thiamine.HCL;
0.100 g of Pyridoxine.HCL; and 0.400 g of Glycine in 875.00 ml of
polished D-I H₂O in sequence.
Bring up to volume with polished D-I H₂O. Make in 400 ml portions.
Thiamine.HCL & Pyridoxine.HCL are in Dark Desiccator. Store for one
month, unless contamination or precipitation occurs, then make fresh
stock.
Total Volume (L) = 1.00

288 J

| | | |
|---|---|---|
| D-I H₂O | 950.000 | Ml |
| MS Salts | 4.300 | g |
| Myo-Inositol | 0.100 | g |
| MS Vitamins Stock Solution ## | 5.000 | ml |
| Zeatin .5 mg/ml | 1.000 | ml |
| Sucrose | 60.000 | g |
| Gelrite @ | 3.000 | g |
| Indoleacetic Acid 0.5 mg/ml # | 2.000 | ml |
| 0.1 mM Abscisic Acid | 1.000 | ml |
| Bialaphos 1 mg/ml # | 3.000 | ml |

Directions:
@ = Add after bringing up to volume
Dissolve ingredients in polished D-I H₂O in sequence
Adjust to pH 5.6
Bring up to volume with polished D-I H₂O after adjusting pH
Sterilize and cool to 60° C.
Add 3.5 g/L of Gelrite for cell biology.
## = Dissolve 0.100 g of Nicotinic Acid; 0.020 g of Thiamine.HCL;
0.100 g of Pyridoxine.HCL; and 0.400 g of Glycine in 875.00 ml of
polished D-I H₂O in sequence.
Bring up to volume with polished D-I H₂O. Make in 400 ml portions.
Thiamine.HCL & Pyridoxine.HCL are in Dark Desiccator. Store for one
month, unless contamination or precipitation occurs, then make fresh
stock.
Total Volume (L) 1.00

560 R

| | | |
|---|---|---|
| D-I Water, Filtered | 950.000 | ml |
| CHU (N6) Basal Salts (SIGMA C-1416) | 4.000 | g |
| Eriksson's Vitamin Mix (1000X SIGMA-1511 | 1.000 | ml |
| Thiamine.HCL 0.4 mg/ml | 1.250 | ml |
| Sucrose | 30.000 | g |
| 2,4-D 0.5 mg/ml | 4.000 | ml |
| Gelrite @ | 3.000 | g |
| Silver Nitrate 2 mg/ml # | 0.425 | ml |
| Bialaphos 1 mg/ml # | 3.000 | ml |

Directions:
@ = Add after bringing up to volume
# = Add after sterilizing and cooling to temp.
Dissolve ingredients in D-I H₂O in sequence
Adjust to pH 5.8 with KOH
Bring up to volume with D-I H₂O
Sterilize and cool to room temp.
Total Volume (L) = 1.00

560 Y

| | | |
|---|---|---|
| D-I Water, Filtered | 950.000 | ml |
| CHU (N6) Basal Salts (SIGMA C-1416) | 4.000 | g |
| Eriksson's Vitamin Mix (1000X SIGMA-1511 | 1.000 | ml |
| Thiamine.HCL 0.4 mg/ml | 1.250 | ml |
| Sucrose | 120.000 | g |
| 2,4-D 0.5 mg/ml | 2.000 | ml |
| L-Proline | 2.880 | g |
| Gelrite @ | 2.000 | g |
| Silver Nitrate 2 mg/ml # | 4.250 | ml |

Directions:
@ = Add after bringing up to volume
# = Add after sterilizing and cooling to temp.
Dissolve ingredients in D-I H₂O in sequence
Adjust to pH 5.8 with KOH
Bring up to volume with D-I H₂O
Sterilize and cool to room temp.
** Autoclave less time because of increased sucrose**
Total Volume (L) = 1.00

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

That which is claimed is:

1. An isolated nucleic acid molecule selected from the group consisting of:
a) a nucleic acid molecule comprising a nucleotide sequence that encodes a polypeptide that confers disease resistance to a plant, said sequence having at least 85% sequence identity to the sequence set forth in SEQ ID NO: 34, 35, or a complement thereof;
b) a nucleic acid molecule comprising a fragment of at least 300 contiguous nucleotides of the sequence set forth in SEQ ID NO: 34, 35, or a complement thereof, wherein said fragment encodes a polypeptide that confers disease resistance to a plant;
c) a nucleic acid molecule that encodes a polypeptide comprising the amino acid sequence set forth in SEQ ID NO: 36;
d) a nucleic acid molecule that encodes a fragment of a polypeptide comprising the amino acid sequence of SEQ ID NO: 36, wherein the fragment retains the ability to confer disease resistance to a plant and comprises at least 100 contiguous amino acids of SEQ ID NO: 36;
e) a nucleic acid molecule that encodes a polypeptide that confers disease resistance to a plant, wherein the nucleic acid molecule hybridizes to a sequence of a), b), c), d), or a complement thereof under stringent conditions, said stringent conditions comprising hybridization in 40% formamide, 1.0 M NaCl, 1% SDS at 37° C., followed by washing with 1×SSC at 55° C.; and
f) a nucleic acid molecule comprising a fragment of at least 50 contiguous nucleotides of SEQ ID NO: 34, 35, or a complement thereof, wherein said fragment hybridizes to a sequence of a), b), c), d), or a complement thereof under stringent conditions, said stringent conditions comprising hybridization in 40% formamide, 1.0 M NaCl, 1% SDS at 37° C., followed by washing with 1×SSC at 55° C.

2. A DNA construct comprising a nucleotide sequence of claim 1 operably linked to a promoter that drives expression in a plant cell.

3. A vector comprising the DNA construct of claim 2.

4. A plant cell having stably incorporated in its genome the DNA construct of claim 2.

5. A plant having stably incorporated in its genome the DNA construct of claim 2.

6. A method for creating or enhancing disease resistance in a plant, said method comprising transforming said plant with a DNA construct comprising a nucleic acid molecule operably linked to a promoter that drives expression of a coding sequence in a plant cell and regenerating stably transformed plants, wherein said nucleic acid molecule is selected from the group consisting of:
a) a nucleic acid molecule comprising a nucleotide sequence that encodes a polypeptide that confers disease resistance to a plant, said sequence having at least 85% sequence identity to the sequence set forth in SEQ ID NO: 34, 35, or a complement thereof;
b) a nucleic acid molecule comprising a fragment of at least 300 contiguous nucleotides of the sequence set forth in SEQ ID NO: 34, 35, or a complement thereof, wherein said fragment encodes a polypeptide that confers disease resistance to a plant;
c) a nucleic acid molecule that encodes a polypeptide comprising the amino acid sequence set forth in SEQ ID NO: 36;
d) a nucleic acid molecule that encodes a fragment of a polypeptide comprising the amino acid sequence of SEQ ID NO: 36, wherein the fragment retains the ability to confer disease resistance to a plant and comprises at least 100 contiguous amino acids of SEQ ID NO: 36; and
e) a nucleic acid molecule that encodes a polypeptide that confers disease resistance to a plant, wherein the nucleic acid molecule hybridizes to a sequence of a), b), c), d), or a complement thereof under stringent conditions, said stringent conditions comprising hybridization in 40% formamide, 1.0 M NaCl, 1% SDS at 37° C., followed by washing with 1×SSC at 55° C.

7. The method of claim 6, wherein said plant is a dicot.

8. The method of claim 6, wherein said plant is a monocot.

9. The method of claim 8, wherein said monocot is selected from the group consisting of maize, sorghum, barley, rice, and wheat.

10. The method of claim 6, wherein said promoter is a constitutive promoter.

11. The method of claim 6, wherein said promoter is an inducible promoter.

12. A plant stably transformed with a DNA construct comprising a nucleic acid molecule operably linked to a promoter that drives expression of a coding sequence in a plant cell, wherein said nucleic acid molecule is selected from the group consisting of:
a) a nucleic acid molecule comprising a nucleotide sequence that encodes a polypeptide disease resistance activity, said sequence having at least 85% sequence identity to the sequence set forth in SEQ ID NO: 34, 35, or a complement thereof;
b) a nucleic acid molecule comprising a fragment of at least 300 contiguous nucleotides of the sequence set forth in SEQ ID NO: 34, 35, or a complement thereof, wherein said fragment encodes a polypeptide that confers disease resistance to a plant;
c) a nucleic acid molecule that encodes a polypeptide comprising the amino acid sequence set forth in SEQ ID NO: 36;
d) a nucleic acid molecule that encodes a fragment of a polypeptide comprising the amino acid sequence of SEQ ID NO: 36, wherein the fragment retains the ability to confer disease resistance to a plant and comprises at least 100 contiguous amino acids of SEQ ID NO: 36; and
e) a nucleic acid molecule which encodes a polypeptide that confers disease resistance to a plant, wherein the nucleic acid molecule hybridizes to a sequence of a), b), c), d), or a complement thereof under stringent conditions, said stringent conditions comprising hybridization in 40% formamide, 1.0 M NaCl, 1% SDS at 37° C., followed by washing with 1×SSC at 55° C.

13. The plant of claim 12, wherein said plant is a dicot.

14. The plant of claim 12, wherein said plant is a monocot.

15. The plant of claim 14, wherein said monocot is selected from the group consisting of maize, sorghum, barley, rice, and wheat.

16. The plant of claim 12, wherein said promoter is a constitutive promoter.

17. The plant of claim 12, wherein said promoter is an inducible promoter.

18. Seed of the plant of claim 12.

19. Seed of the plant of claim 13.

20. Seed of the plant of claim 14.

21. Seed of the plant of claim 15.
